(19) United States
(12) Patent Application Publication
(10) Pub. No.: US 2008/0015138 A1
(43) Pub. Date: Jan. 17, 2008

(54) Metal binding compounds, metal binding compositions, and their uses

(75) Inventors: Paul Hamilton, Cary, NC (US); Wayne F. Beyer, Bahama, NC (US)

Correspondence Address: AFFINERGY, INC., P.O. BOX 14650, DURHAM, NC, 27709, US

(73) Assignee: AFFINERGY, INC., Research Triangle Park, NC (US)

(21) Appl. No.: 11/776,677

(22) Filed: Jul. 12, 2007

Related U.S. Application Data
(60) Provisional application No. 60/831,277, filed on Jul. 17, 2006.

(51) Int. Cl. A61K 38/00; C07H 19/04; C07K 2/00; C07K 4/00

(52) U.S. Cl. 514/2; 106/287.25; 428/458; 530/300; 530/326; 530/327; 530/328; 530/329; 530/350; 536/22.1

(57) ABSTRACT

Compositions are provided comprising a family of peptides having binding specificity for metal, and their use to produce coating compositions. The coating compositions are used to deliver a pharmaceutically active agent to metal, and are used in methods related to metal implants, metal repair, and metal-related diseases.

### GRANT STATEMENT

This invention was made in part from government support under Grant No. 1R43AR051264-01A1 from the National Institute of Arthritis and Musculoskeletal and Skin Diseases. Thus, the U.S. Government has certain rights in the invention.

### FIELD OF THE INVENTION

The present invention relates to metal binding compounds, metal binding compositions comprised of the metal binding compounds, and methods of use thereof such as in industrial, medical, and pharmaceutical applications.

### BACKGROUND OF THE INVENTION

Metal binding peptides have been described as having utility in many different applications including, but not limited to: metal ion affinity chromatography to purify proteins (see, e.g., published application US 2006/0030007); in bioremediation to bind to metal ions or metal-containing compounds; in medicine, such as to inhibit the formation or accumulation of reactive oxygen species in vivo, thereby reducing tissue and cellular damage caused by reactive oxygen species (see, e.g., published application US 2005/0215468; in industrial applications, such as corrosion inhibitors (see, e.g., Zuo et al., Appl. Microbiol. Biotechnol. (2005) 68:505-509); and in medicine, such as a component in interfacial biomaterials or coatings for medical devices to deliver one or more pharmaceutically active agents at the metal surface of the medical device coated by the coating (see e.g., published application US 2006/0051395; assigned to the present assignee).

Thus, there is a need for novel metal-binding peptides, particularly having improved properties, such as, for example, higher binding affinities for metal.

### SUMMARY OF THE INVENTION

In one aspect of this invention, provided are metal-binding peptides of a unique family comprising a metal binding motif (or “metal binding domain”) containing a plurality of one or more triplets of specific amino acids, and wherein each triplet in a plurality of triplets is optimally spaced between the one or more adjacent triplets, in unexpectedly providing high binding affinity to metal, more preferably as a surface (e.g., containing a series or plurality of metal ions) as compared to a single metal ion.

In another aspect of the present invention, provided are peptides containing a metal binding motif showing a structure and function relationship comprising a conserved set of triplets of cationic amino acids, a triplet optimally being separated by two amino acids from an adjacent triplet, in providing unexpectedly higher binding affinity to metal.

In one embodiment of the present invention, provided are metal binding peptides having the formula: (Xaa)ₘZ₁(Xaa)ⱼZ₂(Xaa)ₙ (SEQ ID NO:1), wherein Xaa is an amino acid, for example, one of the 20 naturally occurring amino acids found in proteins in either the L or D form of chiral amino acids or a modified amino acid, except that Xaa is an amino acid other than lysine or histidine when occurring between two Z (e.g., Xaa of the amino acid sequence Z₁(Xaa)ⱼZ₂ is not lysine or histidine); Z is a triplet of amino acids consisting of at least one histidine residue and at least one lysine residue, no other amino acids other than histidine and lysine residues, but no more than two histidine residues or no more than two lysine residues (e.g., KHK, HKH, KKH, HKK, KHH); m is from 0 to 50; n is from 0 to 50; j is from 0 to 5, and more preferably from 2 to 4, and most preferably, 2; and wherein more preferably Z is one of HKH, KKH, or KHK, and most preferably, at least one of Z (e.g., either Z₁ or Z₂, or both of Z₁ and Z₂, in the amino acid sequence Z₁(Xaa)ⱼZ₂) is KHK. Either or both of (Xaa)ₘ and (Xaa)ₙ may comprise from 0 to no more than 10 Z. For example, where j is 2 and n is 50, and (Xaa)₅₀ consists of 10 Z, then (Xaa)₅₀ may consist of an amino acid sequence of

| | |
|---|---|
| XaaXaaZXaaXaaZXaaXaaZXaaXaaZXaaXaaZXaaXaaZXaaXaaZXaaXaaZXaaXaaZXaaXaaZ; | (SEQ ID No: 102) |

In certain examples of a metal binding peptide according to the present invention, the peptide comprises no less than 7 amino acids to no more than about 100 amino acids, preferably from 8 amino acids to about 30 amino acids, and more preferably from 8 amino acids to about 15 amino acids, and comprises an amino acid sequence having a metal binding domain selected from the group consisting of Z₁(Xaa)ⱼZ₂ (SEQ ID NO:2), Z₁(Xaa)ⱼZ₂(Xaa)ⱼZ (SEQ ID NO:3), and a combination thereof. In certain examples of this embodiment, the metal binding domain is

| | |
|---|---|
| KHKXaaXaaKHK, | (SEQ ID NO: 4) |
| HKHXaaXaaHKH, | (SEQ ID NO: 5) |
| KKHXaaXaaKKH, | (SEQ ID NO: 6) |
| KHKXaaXaaHKH, | (SEQ ID NO: 7) |
| HKHXaaXaaKHK, | (SEQ ID NO: 8) |
| KHKXaaXaaKHKXaaXaaKHK, | (SEQ ID NO: 9) |
| HKHXaaXaaHKHXaaXaaHKH; | (SEQ ID NO: 10) |

and in other examples of this embodiment, the metal binding domain is HKHXaaXaaKKH (SEQ ID NO:11), KKHXaaXaaKHK (SEQ ID NO:12), KKHXaaXaaHKH (SEQ ID NO:13), KHKXaaXaaKKH (SEQ ID NO:14), KHKXaaXaaHKHXaaXaaKKH (SEQ ID NO:15), KHKXaaXaaKKHXaaXaaHKH (SEQ ID NO:16), KHKXaaXaaHKHXaaXaaKHK (SEQ ID NO:17), KHKXaaXaaKHKXaaXaaHKH (SEQ ID NO:18), KHKXaaXaaKKHXaaXaaKHK (SEQ ID NO:19), KHKXaaXaaKHKXaaXaaKKH (SEQ ID NO:20), KHKXaaXaaKKHXaaXaaKKH (SEQ ID NO:21), KHKXaaXaaHKHXaaXaaHKH (SEQ ID NO:22), HKHXaaXaaHKHXaaXaaKKH (SEQ ID NO:23), HKHXaaXaaKKHXaaXaaHKH (SEQ ID NO:24), HKHXaaXaaHKHXaaXaaKHK (SEQ ID NO:25), HKHXaaXaaKHKXaaXaaHKH (SEQ ID NO:26), HKHXaaXaaKHKXaaXaaKHK (SEQ ID NO:27), HKHXaaXaaKHKXaaXaaHKH (SEQ ID NO:28), HKHXaaXaaKHKXaaXaaKKH (SEQ ID NO:29), HKHXaaXaaKKHXaaXaaKKH (SEQ ID NO:30), HKHXaaXaaKKHXaaXaaKHK (SEQ ID NO:31), KKHXaaXaaHKHXaaXaaKKH (SEQ ID NO:32), KKHXaaXaaKKHXaaXaaHKH (SEQ ID NO:33), KKHXaaXaaHKHXaaXaaKHK (SEQ ID NO:34), KKHXaaXaaKHKXaaXaaHKH (SEQ ID NO:35), KKHXaaXaaKHKXaaXaaKHK (SEQ ID NO:36), KKHXaaXaaKHKXaaXaaHKH (SEQ ID NO:37), KKHXaaXaaKHKXaaXaaKKH (SEQ ID NO:38), KKHXaaXaaKKHXaaXaaKKH (SEQ ID NO:39), KKHXaaXaaHKHXaaXaaHKH (SEQ ID NO:40), KKHXaaXaaKKHXaaXaaKHK (SEQ ID NO:41), KHKXaaKHK (SEQ ID NO:42), KHKXaaXaaXaaKHK (SEQ ID NO:43), KHKXaaXaaXaaXaaKHK (SEQ ID NO:44), KHKXaaXaaXaaXaaXaaKHK (SEQ ID NO:45); or a combination thereof; with the proviso that Xaa is an amino acid other than lysine or histidine (e.g., Xaa is not lysine, Xaa is not histidine). A preferred metal binding domain may be used to the exclusion of a metal binding domain other than the preferred metal binding domain.

In one embodiment, the peptide may comprise a polymer comprised of a plurality of metal binding domains according to the present invention, wherein each metal binding domain in the polymer may be separated by a contiguous sequence of amino acids ranging from 2 residues to about 50 residues, preferably from about 2 amino acids to about 20 amino acids, and more preferably from 2 amino acids to about 5 amino acids, from the nearest metal binding domain in the amino acid sequence of the peptide. The polymer may be a linear polymer. For example, peptides containing the metal binding domains consisting essentially an amino acid sequence of SEQ ID NOs:18 and 20 are polymers of a peptide containing the metal binding domain consisting essentially of an amino acid sequence of SEQ ID NO:4. Alternatively, the polymer may be a branched polymer. For example, polymers represented by peptides consisting essentially of an amino acid sequence of SEQ ID NOs: 85 and 86 are branched polymers of a peptide consisting essentially of an amino acid sequence of SEQ ID NO:9 (see Example 5 herein).

In another aspect of this invention, provided are a family of peptides that share structure and function, in that the peptides comprise amino acid sequence having at least one metal binding domain comprising a plurality of triplets of amino acids; wherein each triplet consists of at least one but not more than 2 histidine residues, and at least one but not more than two lysine residues; wherein each triplet, comprised within a metal binding domain, is separated by from about 1 amino acid to about five amino acids, and more preferably by two amino acid residues, (other than lysine and/or histidine) from the next closest triplet appearing in the metal binding domain of the amino acid sequence of the peptide; and wherein the family of peptides have binding specificity for metal. Related to this aspect of this invention, provided are nucleotide sequences and vectors encoding such peptides. Also related to this aspect of the invention, provided is a composition comprising a peptide according to the present invention, and a pharmaceutically acceptable carrier.

The invention also provides a method of coating a surface comprised of metal for which peptide of the present invention has binding specificity, the method comprising contacting the peptide, or a composition comprising the peptide, with the surface so that peptide binds to the metal, and coated is the surface. Also provided is a coating composition comprised of a peptide according to the present invention linked to one or more of a peptide having binding specificity for a pharmaceutically active agent, and may further comprise pharmaceutically active agent bound thereto, as will be described in more detail herein. Thus, peptide, or a composition comprising peptide, according to the present invention may be used for delivering and localizing one or more pharmaceutically active agents to a metal, such as a metal surface including, but not limited to, a metal surface of an implant (e.g., medical device). Also provided according to the present invention is a metal surface coated by peptide or peptide-containing composition according to the present invention.

### DETAILED DESCRIPTION OF THE INVENTION

The present invention provides a family of peptides having binding specificity for metal, and a coating composition comprising a peptide according to the present invention, wherein the peptide comprises triplets of a combination of lysine and histidine residues separated by a defined number of amino acids in providing unexpectedly high binding specificity for metal. Also provided are coatings for metal, methods of coating metal, and metal coated with these compositions.

Definition Section While the following terms are believed to be well understood by one of ordinary skill in the art, the following definitions are set forth to facilitate explanation of the invention.

The term “metal” is used herein for purposes of the specification and claims to mean one or more compounds or compositions comprising a metal represented in the Periodic Table, a metal alloy, a metal oxide, a silicon oxide, and bioactive glass. Examples of preferred metals include, but are not limited to, titanium, titanium alloy, stainless steel, aluminum, zirconium alloy metal substrate (e.g., Oxinium™), and cobalt chromium alloy. A preferred type or composition of metal may be used in accordance with the present invention to the exclusion of a type or composition of metal other than the preferred type or composition of metal.

The term “effective amount” is used herein, in referring to a peptide itself, or as part of a coating composition, according to the present invention and for purposes of the specification and claims, to mean an amount sufficient of peptide so as to mediate binding of peptide to the at least one surface of metal in forming a coating; and may further comprise an amount sufficient to promote attachment of a pharmaceutically active agent.

The term “individual”, as used herein for purposes of the specification and claims, refers to either a human or an animal.

The term “pharmaceutically active agent”, as used herein for purposes of the specification and claims, refers to one or more agents selected from the group consisting of growth factor, cells, therapeutic drug, hormone, vitamin, and nucleic acid molecule encoding any of the foregoing, or a nucleic acid molecule having, itself, bioactivity. Hormones include, but are not limited to parathyroid hormone (PTH, including, for example, PTH 1 to PTH 34), and growth hormone. Therapeutic drugs useful in medical applications for treatment or prevention of diseases or disorders include, but are not limited to, chemotherapeutic agents (e.g., methotrexate, cyclophosphamide, taxol, adriamycin, paclitaxel, sirolimus, or other antineoplastic agent), antimicrobials (e.g., antifungal, and/or antibacterial; antibiotics), anti-inflammatory agents (steroidal or nonsteroidal), anti-clotting agents (e.g., aspirin, clopidrogrel, etc.), analgesic agents, anesthetic agents, and nucleic acid molecules that can affect gene regulation such as DNA, antisense RNA, interfering RNAs (e.g., RNAi, siRNA, etc.), RNA fragments (e.g., micro RNAs, modifying RNAs, etc.). Vitamins may include, but are not limited to, vitamin D, and vitamin D derivatives (e.g., 1, 25-dihydroxyvitamin D3, 1α-hydroxyvitamin D2), vitamin A, vitamin C, and vitamin K (e.g., preferably, vitamin K2). A preferred pharmaceutically active agent may be used in accordance with the present invention to the exclusion of a pharmaceutically active agent other than the preferred pharmaceutically active agent.

The term “cells”, as used herein for purposes of the specification and claims, refers to one or more cells or cell types, particular cells of human origin, useful in the present invention, and may include but is not limited to, stem cells, osteoprogenitor stem cells, mesenchymal stem cells, osteocytes, osteoblasts, osteoclasts, periosteal stem cells, metal marrow endothelial cells, endothelial cells, stromal cells, hematopoietic progenitor cells, adipose tissue precursor cells, cord blood stem cells, and a combination thereof. A preferred cell type (preferred cells) may be used in accordance with the present invention to the exclusion of cells other than the preferred cells.

The term “growth factor”, as used herein for purposes of the specification and claims, refers to one or more growth factors or cytokines useful in the present invention, and may include but is not limited to, metal morphogenetic protein (BMP, including the family of BMPs, such as BMP-2, BMP-2A, BMP-2B, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, and BMP-18), transforming growth factor beta (TGF-beta), transforming growth factor alpha (TGF-alpha), vascular endothelial cell growth factor (VEGF, including its variants), epidermal growth factor (EGF), fibroblast growth factor (e.g., basic fibroblast growth factor, acidic fibroblast growth factor, FGF-1 to FGF-23), epidermal growth factor (EGF), insulin-like growth factor (I or II), interleukin-I, interferon, tumor necrosis factor, nerve growth factor, neurotrophins, platelet-derived growth factor (PDGF), heparin-binding growth factor (HBGF), hepatocytic growth factor, keratinocyte growth factor, macrophage colony stimulating factor, growth and differentiation factor (e.g., GDF4 to GDF8), isoforms thereof, biologically active analogs thereof, and a combination thereof. Typically, a biologically analog has an amino acid sequence having from about 1% to about 25% of the amino acids substituted, as compared to the amino acid sequence of the peptide growth factor from which the analog was derived. For peptides less than or equal to 50 amino acids in length, typically a biologically active analog thereof has between 1 and 10 amino acid changes, as compared to the amino acid sequence of the peptide from which the analog was derived. A preferred growth factor may be used in accordance with the present invention to the exclusion of a growth factor other than the preferred growth factor.

The term “time sufficient for binding” generally refers to a temporal duration sufficient for specific binding of a binding domain described herein, and a substrate for which the binding domain has binding specificity, as known to those skilled in the art. Based on the affinity of the peptide forming the binding domain, typically a time sufficient for binding to a substrate ranges from about 5 minutes to no more than 60 minutes.

The term “coating composition” is used herein, in reference to the present invention and for purposes of the specification and claims, to refer to one or more of: a composition comprising peptide according to the present invention, and a component selected from the group consisting of pharmaceutically active agent linked to the peptide, a pharmaceutically acceptable carrier, and a combination thereof; or a composition comprising peptide according to the present invention linked to a peptide of from about 3 amino acids to about 100 amino acids having binding specificity for a pharmaceutically active agent, and which may further comprise pharmaceutically active agent bound thereto.

In an embodiment wherein the composition comprises a peptide having binding specificity for metal linked to a peptide having binding specificity for a pharmaceutically active agent, the respective peptides are coupled together (e.g., by one or more of physically, chemically, synthetically, or biologically (e.g., via recombinant expression)) in such a way that each retains its respective function to bind to the respective molecule for which it has binding specificity. Such coupling may include forming a multimeric molecule having two or more peptides having binding specificity for metal, two or more peptides having binding specificity for a pharmaceutically active agent, and a combination thereof. For example, using standard reagents and methods known in the art of peptide chemistry, two peptides may be coupled via a side chain-to-side chain bond (e.g., where each of the peptides has a side chain amine (e.g., such as the epsilon amine of lysine)), a side chain-to-N terminal bond (e.g., coupling the N-terminal amine of one peptide with the side chain amine of the other peptide), a side chain-to-C-terminal bond (e.g., coupling the C-terminal chemical moiety (e.g., carboxyl) of one peptide with the side chain amine of the other peptide), an N-terminal-to-N-terminal bond, an N-terminal to C-terminal bond, a C-terminal to C-terminal bond, or a combination thereof. In synthetic or recombinant expression, a peptide having binding specificity for metal can be coupled directly to a peptide having binding specificity for a pharmaceutically active agent by synthesizing or expressing both peptides as a single peptide. The coupling of two or more peptides may also be via a linker to form a coating composition.

A coating composition of the present invention comprises the at least one peptide having binding specificity for metal according to the present invention in an amount effective to mediate the binding of the coating composition to the metal surface to be coated. Thus, peptide by itself or as a component in a coating composition provides for targeting and localizing a pharmaceutically active agent to metals. In one embodiment, the coating composition comprises at least one peptide having binding specificity for metal and at least one peptide having binding specificity for a pharmaceutically active agent, wherein the at least one peptide having binding specificity for metal and the at least one peptide having binding specificity for a pharmaceutically active agent are coupled together. In another embodiment, the coating composition comprises at least one peptide having binding specificity for metal, and at least one peptide having binding specificity for a pharmaceutically active agent, wherein the at least one peptide having binding specificity for metal and the at least one peptide having binding specificity for a pharmaceutically active agent are coupled together, and wherein the at least one peptide having binding specificity for a pharmaceutically active agent is bound (preferably, noncovalently) to a pharmaceutically active agent for which it has binding specificity. In a preferred embodiment, a linker is used to couple the at least one peptide having binding specificity for metal and the at least one peptide having binding specificity for a pharmaceutically active agent.

The at least one peptide having binding specificity for metal according to the present invention may be comprised of peptide having binding specificity for metal (e.g., peptide comprising one amino acid sequence, such as consisting essentially of SEQ ID NO:9), or may be comprised of two or more peptides (e.g., linked by a multi-branched linker, or each as separate components of the composition) comprising either (a) the same amino acid sequence (e.g., consisting essentially of SEQ ID NO:9) or (b) two or more amino acid sequences (e.g., one peptide comprising the amino acid sequence consisting essentially of SEQ ID NO:9, another peptide comprising the amino acid sequence consisting essentially of SEQ ID NO:10, etc.). The at least one peptide having binding specificity for a pharmaceutically active agent may be comprised of peptide having binding specificity for a single type of pharmaceutically active agent (e.g., peptide having binding specificity for cells), or may be comprised of two or more peptides comprising either (a) the same binding specificity (e.g., each peptide binding the same growth factor or family of related growth factors) or (b) two or more amino acid sequences having different binding specificities (e.g., one peptide having a binding specificity for a growth factor, and another peptide having binding specificity for a hormone, etc).

The term “linker” is used, for purposes of the specification and claims, to refer to a compound or moiety that acts as a molecular bridge to couple at least two separate molecules (e.g., with respect to the present invention, coupling at least one peptide having binding specificity for metal to at least one peptide having binding specificity for a pharmaceutically active agent). Thus, for example, one portion of the linker binds to at least one peptide having binding specificity for metal according to the present invention, and another portion of the linker binds to at least one peptide having binding specificity for a pharmaceutically active agent. As known to those skilled in the art, and using methods known in the art, the two peptides may be coupled to the linker in a step-wise manner, or may be coupled simultaneously to the linker, to form a coating composition according to the present invention. There is no particular size or content limitations for the linker so long as it can fulfill its purpose as a molecular bridge, and that the binding specificity of each peptide in a coating composition is substantially retained.

Linkers are known to those skilled in the art to include, but are not limited to, chemical compounds (e.g., chemical chains, compounds, reagents, and the like). The linkers may include, but are not limited to, homobifunctional linkers and heterobifunctional linkers. Heterobifunctional linkers, well known to those skilled in the art, contain one end having a first reactive functionality (or chemical moiety) to specifically link a first molecule, and an opposite end having a second reactive functionality to specifically link to a second molecule. It will be evident to those skilled in the art that a variety of bifunctional or polyfunctional reagents, both homo- and hetero-functional (such as those described in the catalog of the Pierce Chemical Co., Rockford, Ill.), amino acid linkers (typically, a short peptide of between 3 and 15 amino acids, and often containing amino acids such as glycine, and/or serine), and polymers (e.g., polyethylene glycol or other polymer as described herein) may be employed as a linker with respect to the present invention. In one embodiment, representative peptide linkers comprise multiple reactive sites (or “reactive functionalities”) to be coupled to a binding domain (e.g., polylysines, polyornithines, polycysteines, polyglutamic acid and polyaspartic acid) or comprise substantially inert peptide linkers (e.g., lipolyglycine, polyserine, polyproline, polyalanine, and other oligopeptides comprising alanyl, serinyl, prolinyl, or glycinyl amino acid residues). In some embodiments wherein amino acid linker is chosen, the coating composition may be synthesized to be a single, contiguous peptide comprising a peptide having binding specificity for metal according to the present invention, a linker, and a peptide having binding specificity for a pharmaceutically active agent. Thus, the linker attachment is simply via the bonds of the single contiguous peptide.

Suitable polymeric linkers are known in the art, and can comprise a synthetic polymer or a natural polymer. Representative synthetic polymers include but are not limited to polyethers (e.g., poly(ethylene glycol) (“PEG”)), polyesters (e.g., polylactic acid (PLA) and polyglycolic acid (PGA)), polyamines, polyamides (e.g., nylon), polyurethanes, polymethacrylates (e.g., polymethylmethacrylate; PMMA), polyacrylic acids, polystyrenes, polyhexanoic acid, flexible chelators such as EDTA, EGTA, and other synthetic polymers which preferably have a molecular weight of about 20 daltons to about 1,000 kilodaltons. Representative natural polymers include but are not limited to hyaluronic acid, alginate, chondroitin sulfate, fibrinogen, fibronectin, albumin, collagen, calmodulin, and other natural polymers which preferably have a molecular weight of about 200 daltons to about 20,000 kilodaltons (for constituent monomers). Polymeric linkers can comprise a diblock polymer, a multi-block copolymer, a comb polymer, a star polymer, a dendritic or branched polymer, a hybrid linear-dendritic polymer, a branched chain comprised of lysine, or a random copolymer. A linker can also comprise a mercapto(amido)carboxylic acid, an acrylamidocarboxylic acid, an acrlyamido-amidotriethylene glycolic acid, 7-aminobenzoic acid, and derivatives thereof. Linkers may also utilize copper-catalyzed azide-alkyne cycloaddition (e.g., “click chemistry”) or any other methods well known in the art. Linkers are known in the art and include linkers that can be cleaved, and linkers that can be made reactive toward other molecular moieties or toward themselves, for cross-linking purposes.

Depending on such factors as the molecules to be linked, and the conditions in which the linking is performed, the linker may vary in length and composition for optimizing such properties as preservation of biological function, stability, resistance to certain chemical and/or temperature parameters, and of sufficient stereo-selectivity or size. For example, the linker should not significantly interfere with the ability of a coating composition to sufficiently bind, with appropriate avidity for the purpose, to a metal for which it has specificity according to the present invention, or the ability of a coating composition to sufficiently bind, with appropriate avidity for the purpose, to a pharmaceutically active agent for which it has specificity. A preferred linker may be a molecule which may have activities which enhance or complement the effect of the coating composition of the present invention. A preferred linker may be used in the present invention to the exclusion of a linker other than the preferred linker.

The terms “binds specifically” or “binding specificity”, and like terms used herein, are interchangeably used, for the purposes of the specification and claims, to refer to the ability of a peptide (as described herein) to have a binding affinity that is greater for one target molecule selected to be bound (the latter, “target surface material”) over another molecule or surface material (other than the target molecule or target surface material); e.g., an affinity for a given substrate in a heterogeneous population of other substrates which is greater than, for example, that attributable to non-specific adsorption. For example, a peptide has binding specificity for metal when the peptide demonstrates preferential binding to metal, as compared to binding to a component other than metal (e.g., a polymer). Such preferential binding may be dependent upon the presence of a particular conformation, structure, and/or charge on or within the peptide, and/or metal for which it has binding specificity.

In some embodiments, a peptide that binds specifically to a particular surface, material or composition binds at least 10%, 20%, 30%, 40%, 50%, 60%, 70%, 80%, 90%, 100%, 200%, 300%, 400%, 500%, or a higher percentage, than the peptide binds to an appropriate control such as, for example, a different material or surface, or a protein typically used for such comparisons such as bovine serum albumin. For example, binding specificity can determined by an assay in which quantitated is a signal (e.g., fluorescence, or calorimetric) representing the relative amount of binding between a peptide and metal, as compared to peptide and materials other than metal. In a preferred embodiment, a peptide has a binding specificity that is characterized by a relative binding affinity as measured by an EC50 of 1 μM or less, and more preferably less than 0.1 μM. The EC50 can be determined using any number of methods known in the art, such as by generating a concentration response curve from a binding assay in which the concentration of the peptide is titered with a known amount of the substrate for which the peptide has binding specificity (see, for example, methods described in Examples 1 & 2 herein). In such case, the EC50 represents the concentration of peptide producing 50% of the maximal binding observed for that peptide in the assay.

The term “peptide” is used herein, for the purposes of the specification and claims to refer to an amino acid chain of no less than about 3 amino acids and no more than about 200 amino acid residues in length, wherein the amino acid chain may include naturally occurring amino acids, synthetic amino acids, genetically encoded amino acids, non-genetically encoded amino acids, and combinations thereof; however, specifically excluded from the scope and definition of “peptide” herein is an antibody. Preferably, a peptide comprising a metal binding domain according to the present invention comprises a contiguous sequence of no less than 8 amino acids and no more than about 100 amino acids in length, multimers of the peptide (e.g., linking more than one peptide to a branched polymeric linker using methods known in the art), or polymers of a peptide according to the present invention. A polymer of a peptide according to the present invention may comprise at least two, and preferably more than two, metal binding motifs according to the present invention in an amino acid sequence of a polypeptide, wherein each metal binding motif is separated by a sequence of contiguous amino acids ranging from 1 amino acids to about 100 amino acids (and more preferably, from a minimum of at least 3 amino acid residues to a maximum of about 10 amino acid residues, or 15 amino acid residues, or 20 amino acid residues, or more) from the next nearest metal binding motif in the amino acid sequence of the polypeptide. A peptide in accordance with the present invention may be produced by chemical synthesis, recombinant expression, biochemical or enzymatic fragmentation of a larger molecule, chemical cleavage of larger molecule, a combination of the foregoing or, in general, made by any other method in the art, and preferably isolated. The term “isolated” means that the peptide is substantially free of components which have not become part of the integral structure of the peptide itself; e.g., such as substantially free of cellular material or culture medium when produced by recombinant techniques, or substantially free of chemical precursors or other chemicals when chemically synthesized or produced using biochemical or chemical processes. A preferred peptide may be used in the present invention to the exclusion of a peptide other than the preferred peptide.

Peptides can include L-form amino acids, D-form amino acids, or a combination thereof. Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; ornithine; and 3-(3,4-dihydroxyphenyl)-L-alanine (“DOPA”). Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. In a preferred embodiment, and in a coating composition according to the present invention, the at least one peptide having binding specificity for metal may be modified, such as having an N-terminal amino acid, a C-terminal amino acid, or a combination thereof, wherein such amino acid is a non-genetically encoded amino acid that enhances the binding avidity (strength of binding interactions) of the peptide to metal. Such amino acids can be incorporated into a peptide by standard methods known in the art for solid phase and/or solution phase synthesis. For example, in one embodiment, from about one to about three residues of DOPA, a hydroxy-amino acid (e.g., one or more of hydroxylysine, allo-hydroxylysine, hydroxyproline, and the like) or a combination thereof, is added as terminal amino acids of an amino acid sequence of a peptide during synthesis, wherein the peptide is used in the coating composition according to the present invention for enhancing the strength of the binding interactions (e.g., via electrostatic or ionic interactions) between the coating composition and the at least one metal surface to be coated.

A peptide according to the present invention may be modified, such as by addition of chemical moieties to one or more amino acid termini, and side chains; or substitutions, insertions, and deletions of amino acids; where such modifications provide for certain advantages in its use, and provided that the peptide contain a metal binding motif of the present invention. Thus, the term “peptide” encompasses any of a variety of forms of peptide derivatives including, for example, amides, conjugates with proteins, cyclone peptides, polymerized peptides, conservatively substituted variants, analogs, fragments, chemically modified peptides, and peptide mimetics. Any peptide modification that has desired binding characteristics of the family of peptides according to the present invention can be used in the practice of the present invention, provided that the modified peptide has a metal binding domain according to the present invention. For example, a chemical group, added to the N-terminal amino acid of a synthetic peptide to block chemical reactivity of that amino terminus of the peptide, comprises an N-terminal group. Such N-terminal groups for protecting the amino terminus of a peptide are well known in the art, and include, but are not limited to, lower alkanoyl groups, acyl groups, sulfonyl groups, and carbamate forming groups. Preferred N-terminal groups may include acetyl, Fmoc, and Boc. A chemical group, added to the C-terminal amino acid of a synthetic peptide to block chemical reactivity of that carboxy terminus of the peptide, comprises a C-terminal group. Such C-terminal groups for protecting the carboxy terminus of a peptide are well known in the art, and include, but are not limited to, an ester or amide group. Terminal modifications of a peptide are often useful to reduce susceptibility by proteinase digestion, and to therefore prolong a half-life of peptides in the presence of biological fluids where proteases can be present. Optionally, a peptide, as described herein, can comprise one or more amino acids that have been modified to contain one or more chemical groups (e.g., reactive functionalities such as fluorine, bromine, or iodine) to facilitate linking the peptide to a linker molecule. As used herein, the term “peptide” also encompasses a peptide wherein one or more of the peptide bonds are replaced by pseudopeptide bonds including but not limited to a carba bond (CH₂—CH₂), a depsi bond (CO—O), a hydroxyethylene bond (CHOH—CH₂), a ketomethylene bond (CO—CH₂), a methylene-oxy bond (CH₂—O), a reduced bond (CH₂—NH), a thiomethylene bond (CH₂—S), an N-modified bond (—NRCO—), and a thiopeptide bond (CS—NH).

Peptides which are useful in a coating composition or method of using the coating composition according to the present invention also include peptides having one or more substitutions, additions and/or deletions of residues relative to the sequence of an exemplary peptide disclosed in SEQ ID NOs:1-45, 70-79, and 81-86 herein, so long as the peptide maintains a metal binding domain according to the present invention and properties of the original exemplary peptide are substantially retained. Thus, the present invention includes peptides that differ from the exemplary sequences disclosed herein by about 1, 2, 3, 4, 5, 6, 7, 8, 9, or 10 amino acids (depending on the length of the exemplary peptide disclosed herein), and that share sequence identity with the exemplary sequences disclosed herein of at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater sequence identity. Sequence identity may be calculated manually or it may be calculated using a computer implementation of a mathematical algorithm, for example, GAP, BESTFIT, BLAST, FASTA, and TFASTA, or other programs or methods known in the art. Alignments using these programs can be performed using the default parameters.

A peptide having an amino acid sequence substantially identical to a sequence of an exemplary peptide disclosed herein may have one or more different amino acid residues as a result of substituting an amino acid residue in the sequence of the exemplary peptide with a functionally similar amino acid residue (a “conservative substitution”); provided that the conservatively substituted peptide contains a metal binding domain according to the present invention. Examples of conservative substitutions include the substitution of one non-polar (hydrophobic) residue such as isoleucine, valine, leucine or methionine for another; the substitution of one aromatic residue such as tryptophan, tyrosine, or phenylalanine for another; the substitution of one polar (hydrophilic) residue for another such as between arginine and lysine, between glutamine and asparagine, between threonine and serine; the substitution of one basic residue such as lysine, arginine or histidine for another; or the substitution of one acidic residue such as aspartic acid or glutamic acid for another.

In yet another embodiment of the present invention, a peptide according to the present invention may be described as consisting essentially of a peptide (and/or its amino acid sequence) useful in the present invention. When used herein in reference to the present invention and for purposes of the specification and claims, the terminology “consisting essentially of” refers to a peptide which includes a metal binding motif as described herein, and amino acid sequence of the peptides described herein along with conservative substitutions thereof and modifications thereof (as described previously herein in more detail). Preferably, such peptide has at least 70% identity, and preferably at least 95% identity, to an amino acid sequence disclosed herein (e.g., any one of SEQ ID NOs:1-45, 70-79, and 81-86 while containing a metal binding motif according to the present invention, along with additional amino acids at the carboxyl and/or amino terminal ends (e.g., ranging from about 1 to about 50 additional amino acids at one end or at each of both ends; see, e.g., SEQ ID NO:1) which maintains the primary activity of the peptides as metal binding, as described herein. Thus, as a non-limiting example, a peptide or “consisting essentially of” any one of the amino acid sequences illustrated as SEQ ID NOs:2-45, 70-79, & 81-86 will possess the activity of binding metal with binding specificity (a “metal binder”) and will contain a metal binding motif, as provided herein; and will not possess any characteristics which constitutes a material change to the basic and novel characteristics of the peptide to function as a metal binder (e.g., thus, in the foregoing example, a full length naturally occurring polypeptide, or a genetically engineered polypeptide, which has a primary activity other than as a metal binder described herein, and which contains the amino acid sequence containing a metal binding domain described in the present invention, would not constitute a peptide “consisting essentially of” a peptide described in the present invention).

The term “pharmaceutically acceptable carrier”, when used herein for purposes of the specification and claims, means a carrier medium that does not significantly alter the biological activity of the active ingredient (e.g., a peptide or coating composition according to the present invention) to which it is added. Examples of such a carrier medium include, but are not limited to, aqueous solutions, aqueous or non-aqueous solvents, suspensions, emulsions, gels, pastes, and the like. As known to those skilled in the art, a suitable pharmaceutically acceptable carrier may comprise one or substances, including but not limited to, water, buffered water, medical parenteral vehicles, saline, 0.3% glycine, aqueous alcohols, isotonic aqueous buffer; and may further include one or more substances such as water-soluble polymer, glycerol, polyethylene glycol, glycerin, oils, salts such as sodium, potassium, magnesium and ammonium, phosphonates, carbonate esters, fatty acids, saccharides, polysaccharides, glycoproteins (for enhanced stability), excipients, and preservatives and/or stabilizers (to increase shelf-life or as necessary and suitable for manufacture and distribution of the composition).

The terms “implant” or “medical device” are used herein synonymously to generally refer to a structure that is introduced into a human or animal body to ameliorate damage or a disorder or disease, repair or restore a function of a damaged tissue, or to provide a new function. An implant device can be created using any biocompatible material to which a peptide, or peptide-containing composition, according to the present invention can specifically bind as disclosed herein. Representative implants include but are not limited to: hip endoprostheses, artificial joints, jaw or facial implants, dental implants, tendon and ligament replacements, skin replacements, metal replacements and artificial metal screws, metal graft devices, vascular prostheses, heart pacemakers, artificial heart valves, closure devices, breast implants, penile implants, stents, catheters, shunts, nerve growth guides, intraocular lenses, wound dressings, and tissue sealants. Implants are made of a variety of materials that are known in the art and include but are not limited to: a polymer or a mixture of polymers including, for example, polylactic acid, polyglycolic acid, polylactic acid-polyglycolic acid copolymers, polyanhidrides, polyorthoesters, polystyrene, polycarbonate, nylon, PVC, collagen (including, for example, processed collagen such as cross-linked collagen), glycosaminoglycans, hyaluronic acid, alginate, silk, fibrin, cellulose, and rubber; plastics such as polyethylene (including, for example, high-density polyethylene (HDPE)), PEEK (polyetheretherketone), and polytetrafluoroethylene; metals such as titanium, titanium alloy, stainless steel, and cobalt chromium alloy; metal oxides; non-metal oxides; silicon oxides; bioactive glass; ceramic material such as, for example, aluminum oxide, zirconium oxide, and calcium phosphate; other suitable materials such as demineralized metal matrix; and combinations thereof.

### [End of Formal Definition Section]

The present invention provides for a family of peptides having binding specificity for metal; a coating composition comprising a peptide according to the present invention; methods for coating metal with a coating composition according to the present invention; and a metal surface, or an implant (e.g., medical device), coated with a peptide or coating composition according to the present invention; all relating to a peptide containing a metal binding motif according to the present invention. In one embodiment, the coating composition comprises one or more peptides having binding specificity for metal, and may further comprise a pharmaceutically acceptable carrier. Exemplary peptides may be a peptide comprising an amino acid selected from the group consisting of SEQ ID NOs:1 to 45, 70-79, & 81-86, peptide containing a conservative substitution thereof (while retaining a metal binding domain according to the present invention; a “conservatively substituted variant”) and peptide consisting of a modification thereof (while retaining a metal binding domain according to the present invention; a “modified peptide”). In another embodiment, the coating composition comprises at least one peptide having binding specificity for metal, the peptide being coupled to at least one peptide having binding specificity for a pharmaceutically active agent. In another embodiment, the coating composition comprises at least one peptide having binding specificity for metal, the at least one peptide being coupled to at least one peptide having binding specificity for a pharmaceutically active agent having pharmaceutically active agent bound thereto. The coating composition may further comprise a pharmaceutically acceptable carrier. The coating composition is applied to a metal in an amount sufficient to coat the metal, and if further comprising a pharmaceutically active agent, in an amount sufficient to promote the ability of the pharmaceutically active agent to function in its intended pharmaceutical effect (i.e., as known to those skilled in the art to result from the pharmaceutical properties of the pharmaceutically active agent). The present invention is illustrated in the following examples, which are not intended to be limiting.

### EXAMPLE 1

Illustrated in this example are various methods for utilizing phage display technology to produce a metal binding peptide according to the present invention. Many of the peptides comprising the binding domains in a coating composition according to the present invention (i.e., a peptide having binding specificity for metal, and a peptide having binding specificity for a pharmaceutically active agent) were initially developed using phage display technology, followed by peptide design and peptide synthesis to result in improved binding properties.

### Phage Screening and Selections

Phage display technology is well-known in the art, and can be used to try to identify phage-displayed peptides having binding specificity for a certain target substrate used in screening. In general, using phage display, a library of diverse peptides can be presented to a target substrate, and peptides that specifically bind to the substrate can be selected for use as binding domains. Multiple serial rounds of selection, called “panning,” may be used. As is known in the art, any one of a variety of libraries and panning methods can be employed in practicing phage display technology. Panning methods can include, for example, solution phase screening, solid phase screening, or cell-based screening. Once a candidate binding domain is identified, directed or random mutagenesis of the sequence may be used to optimize the binding properties (including one or more of specificity and avidity) of the binding domain.

For example, a variety of different phage display libraries were screened for peptides that bind to a selected target substrate (e.g., a substrate selected to find a binding domain useful in the present invention). The substrate was either bound to or placed in (depending on the selected substrate) a container (e.g., wells of a 96 well microtiter plate, or a microfuge tube). Nonspecific binding sites on the surfaces of the container were blocked with a buffer containing bovine serum albumin (“BSA”; e.g., in a range of from 1% to 10%). The containers were then washed 5 times with a buffer containing buffered saline with Tween™ 20 (“buffer-T”). Each library was diluted in buffer-T and added at a concentration of 10¹⁰ pfu/ml in a total volume of 100 μl. After incubation (in a range of from 1 to 3 hours) at room temperature with shaking at 50 rpm, unbound phage were removed by multiple washes with buffer-T. Bound phage were used to infect E. coli cells in growth media. The cell and phage-containing media was cultured by incubation overnight at 37° C. in a shaker at 200 rpm. Phage-containing supernatant was harvested from the culture after centrifuging the culture. Second and third rounds of selection were performed in a similar manner to that of the first round of selection, using the amplified phage from the previous round as input. To detect phage that specifically bind to the selected substrate, enzyme-linked immunosorbent (ELISA-type) assays were performed using an anti-phage antibody conjugated to a detector molecule, followed by the detection and quantification of the amount of detector molecule bound in the assay. The DNA sequences encoding peptides from the phage that specifically bind to the selected substrate were then determined; i.e., the sequence encoding the peptide is located as an insert in the phage genome, and can be sequenced to yield the corresponding amino acid sequence displayed on the phage surface.

As a specific illustrative example, metal (titanium or stainless steel) was used as a substrate for performing phage selection using several different libraries of phage. Titanium beads and stainless steel beads of approximately 5/32-inch diameter were individually prepared for selections by sequentially washing the beads with 70% ethanol, 40% nitric acid, distilled water, 70% ethanol and, finally, acetone, to remove any surface contaminants. After drying, one metal bead was placed per well of a 96-well polypropylene plate. Non-specific binding sites on the metal beads and the surface of the polypropylene plate were blocked with 1% bovine serum albumin (BSA) in phosphate-buffered saline (PBS). The plate was incubated for 1 hour at room temperature with shaking at 50 rpm. The wells were then washed 5 times with 300 μL of buffer-T.

Each library was diluted in buffer-T and added at a concentration of 10¹⁰ pfu/mL in a total volume of 100 μL. After 3 hours of incubation at room temperature and shaking at 50 rpm, unbound phage were removed by 5 washes of buffer-T. The phage were added directly to E. coli DH5αF′ cells in 2×YT media, and the phage-infected cells were transferred to a fresh tube containing 2×YT media and incubated overnight at 37° C. in a shaker incubator. Phage supernatant was harvested by centrifugation at 8500×g for 10 minutes. Second and third rounds of selection were performed in a similar manner to the first round, using the amplified phage from the previous round as input. Each round of selection was monitored for enrichment of metal binding peptides using ELISA-like assays performed using an anti-M13 phage antibody conjugated to horseradish-peroxidase, followed by the addition of chromogenic agent ABTS (2,2′-azino-bis(3-ethylbenzthiazoline-6-sulphonic acid), and determining a read-out at 405 nm. Libraries that showed enrichment of phage displaying metal binding peptides were plated on a lawn of E. coli cells, and individual plaques were picked and tested for binding to metals (e.g., titanium, stainless steel, etc.). Relative binding strengths of the phage can also be determined by testing serial dilutions of the phage for binding to a metal substrate in an ELISA. For example, serial dilutions of the pooled, display-selected clones were exposed to titanium or steel in an ELISA. The higher dilutions represent more stringent assays for affinity; therefore, phage that yield a signal at higher dilutions represent peptides with higher relative affinity for the particular target metal. Primers against the phage vector sequence that flank the insertion site were used to determine the DNA sequence encoding the peptide for the phage in each group. The sequence encoding the peptide insert was translated to yield the corresponding amino acid sequence displayed on the phage surface.

The DNA sequences encoding peptides isolated on titanium and stainless steel were determined and are shown in Tables 1 and 2, respectively. While typically such phage amino acids adjoining the peptide displayed had no significant contribution to the binding specificity of the peptide, the peptides according to the present invention may also comprise, in their amino acid sequence, such phage amino acids adjoining the peptide at the N-terminus and at the C-terminus (e.g., denoted as ss and sr in Tables 1 & 2).

TABLE 1

Peptide sequences isolated by titanium
selections

| | |
|---|---|
| SEQ ID NO: | Amino acid sequence |

| | |
|---|---|
| 46 | ssHKHPVTPRFFVVEsr |
| 47 | ssCNCYVTPNLLKHKCYKICsr |
| 48 | ssCSHNHHKLTAKHQVAHKCsr |
| 49 | ssCDQNDIFYTSKKSHKSHCsr |
| 50 | ssSSDVYLVSHKHHLTRHNSsr |
| 51 | ssSDKCHKHWYCYESKYGGSsr |
| 52 | HHKLKHQMLHLNGG |
| 53 | GHHHKKDQLPQLGG |

TABLE 2

Peptide sequences isolated by stainless steel
selections

| | |
|---|---|
| SEQ ID NO: | Amino acid sequence |

| | |
|---|---|
| 54 | ssCKHDSEFIKKHVHAVKKCsr |
| 55 | ssCHDHSNKYLKSWKHQQNCsr |
| 56 | ssSYFNLGLVKHNHVRHHDSsr |
| 57 | ssCHHLKHNTHKESKMHHECsr |
| 58 | ssVNKMNRLWEPLsr |

A comparison of the peptides listed in Tables 1 and 2 reveals some common characteristics among the metal-binding peptides that were isolated. Almost all of the peptides are rich in histidine and lysine residues, with most of the peptides having at least five histidine and lysine residues. At first look, the amino acid compositions suggest that the peptides are binding to the oxide surface of the metals via electrostatic interactions between the negatively charged metal surface and the basic amino acids (lysine and histidine). However, arginine, another basic amino acid, is not enriched in the metal-binding peptides discovered by this process. This was the first indication that the interaction between the peptide and the metal surface must be more complex than just a positive charge-negative charge interaction.

### EXAMPLE 2

Peptides according to the present invention may be synthesized using any method known to those skilled in the art including, but not limited to, solid phase synthesis, solution phase synthesis, linear synthesis, and a combination thereof. In this example, peptides were synthesized using standard solid-phase peptide synthesis techniques on a peptide synthesizer using standard Fmoc chemistry. After all residues were coupled, simultaneous cleavage and side chain deprotection was achieved by treatment with a trifluoroacetic acid (TFA) cocktail. Crude peptide was precipitated with cold diethyl ether and purified by high performance liquid chromatography (HPLC) using a linear gradient of water/acetonitrile containing 0.1% TFA. Homogeneity of the synthetic peptides was evaluated by analytical reverse phase-HPLC, and the identity of the peptides was confirmed with mass spectrometry.

### Binding Specificity Characterizations

Relative binding strengths (affinities) of the peptides to metal, also used as a measure of binding specificity, were determined by testing serial dilutions of the peptide for binding to a target substrate comprising metal, as represented by titanium or steel. Plotting the absorbance observed across the concentration range for each peptide sequence yielded a binding curve of the peptides to its target substrate from which can be determined an EC50 (e.g., the concentration of peptide that gives 50% of the maximum signal in the binding curve is used as an estimate of the affinity of the peptide for the target). Preferred are peptides that bind to the selected target substrate (in this case, metal) with binding specificity, preferably with an EC50 of less than or equal to about 1 μM, and more preferably, in the nanomolar range (e.g., <0.1 μM). Thus, in a preferred embodiment, in the methods and compositions according to the present invention, a preferred metal binding domain comprises a peptide demonstrating binding specificity for the selected target substrate metal with an EC50 of less than or equal to about 1 μM, and more preferably, <0.1 μM. A typical binding assay for titanium (note, a different substrate may be substituted for titanium in the assay) may be perofrmed according to the following procedure.

Briefly, 5/32-inch diameter Grade 200 titanium beads were washed by sonication in acetone for 15 minutes, and the beads were allowed to dry. One bead was added to each well of a 96-well polypropylene plate. Two hundred fifty (250) μL of 1% BSA in PBS was added to each well of the plate. The surface of the wells was blocked by incubation for 1 hour at 20° C. with shaking at 500 rpm. The plate was washed three times with 250 μL of buffer-T per well. A 1:3 dilution series of each of the peptides was prepared using PBS as a diluent, starting at a peptide concentration of 20 μM, and going down to 0.0001 μM. A 200 μL sample of each dilution was added to wells of the plate. The plate was incubated for 1 hour at 20° C. with shaking at 500 rpm. The beads were washed three times with 250 μL of buffer-T per well. Two hundred (200) μL of streptavidin-alkaline phosphatase (“streptavidin AP”) reagent, at a dilution of 1:2000 in buffer+1% BSA, was added to each well. The plate was incubated for 30 minutes at room temperature. The beads were washed three times with 250 μL of buffer-T per well. Two hundred (200) μL of color development reagent (PNPP, p-nitrophenol phosphate) was added to each well. After color had developed (10 minutes), the samples were transferred to a clear 96-well plate and the absorbance at 405 nm determined. A binding curve was generated by plotting the absorbance at 405 nm against the peptide concentration (μM).

In comparing binding specificity demonstrated by peptides in Table 1 (consisting of any one of SEQ. ID NOs:47, 48, 49, and 51, and which were biotinylated to facilitate detection and quantification) showed binding to both titanium and stainless steel, with a peptide consisting of SEQ ID NO:47 showing the strongest binding to both metals (with an EC50 of about 800 nM on titanium, and an EC50 of approximately 1 μM on stainless steel). Metal binding was also identified for other metals used clinically as substrates for implants.

### Defining Residues Responsible for Metal Binding

To define which amino acid residues in the peptide were important for metal-binding activity, a series of amino acid substitutions were made based on the amino acid sequences of the peptides illustrated in Table 1. The peptides containing the amino acid substitutions were synthesized, labeled with biotin, and tested for binding to titanium to determine the EC50. Relative titanium-binding strength of each substituted peptide is shown in Table 3.

TABLE 3

Relative binding specificities of substituted
peptides

| | | | |
|---|---|---|---|
| SEQ | | | |
| ID | EC50 | | |
| NO: | (μM) | Sequence | Comment |

| | | | |
|---|---|---|---|
| 59 | 4 | SHKHPVTPRFFVVESK | Parent |
| 60 | 2 | SHKHPVTPRGGVVESK | Replaced FF with GG |
| 61 | 3 | SHKHGGGGRFFVVESK | Replaced PVTP with |
| | | | GGGG |
| 62 | 3 | SHKHPVTPRGGGGESK | Replaced FFVV with |
| | | | GGGG |
| 63 | >50 | SHKHPVTPGFFVVESK | Replaced R with G |
| 64 | >100 | SGGGPVTPRFFVVESK | Replaced HKH with GGG |
| 65 | 0.05 | SHKHPVTPRFFVVYSK | Replaced E with Y |
| 66 | 0.05 | SHKHPVTPRFFVVKSK | Replaced E with K |
| 67 | 0.2 | SHKHPVTPRFFVVVSK | Replaced E with V |
| 68 | 0.6 | SHKHPVTPRFFVVGSK | Replaced E with G |
| 69 | 0.8 | SHKHPVTPRFFVVNSK | Replaced E with N |

The relative affinity of each peptide for binding titanium was compared along with the changes in the amino acid sequence to determine the importance of the various amino acids in binding to metal. From these results, a triplet of amino acid residues, HKH, was determined to play a major role in metal binding. Additionally, the amino acid residue composition contiguous with (adjoining) the triplet of amino acids is not critical for binding to metal.

### Second Generation Metal-Binding Peptides

Based on the titanium-binding affinity results shown in Table 3, a series of synthetic, second-generation peptides were synthesized to further define the elements involved in metal binding, including varying the number (ranging from 0 to 3) of triplets of positively charged amino acids, and the amino acid sequence of triplets of positively charged amino acids. Each peptide was synthesized with an amino acid linker (GSSGK portion of SEQ ID NOs:70-80) to facilitate biotinylation at the C-terminal lysine residue, and detection and quantification in the binding assay. The binding assay was performed using the methods as previously outlined herein The second-generation peptide sequences and the relative binding affinities (EC50) of the peptides for binding to titanium are provided in Table 4.

TABLE 4

Relative binding specificities of second-
generation peptides

| | | |
|---|---|---|
| SEQ ID NO: | Amino acid sequence | EC50 (μM) |

| | | |
|---|---|---|
| 70 | SKKHGGKKHGSSGK | 0.013 |
| 71 | SKHKGGKHKGSSGK | 0.026 |
| 72 | SHKHGGHKHGGHKHGSSGK | 0.035 |
| 73 | SKHKGGHKHGSSGK | 0.045 |
| 74 | SHKHGGKHKGSSGK | 0.060 |
| 75 | SKHKGGGGKHKGSSGK | 0.11 |
| 76 | SHKHGGGGHKHGSSGK | 0.15 |
| 77 | SHKHGGHKHGSSGK | 0.20 |
| 78 | SHHKGGHHKGSSGK- | 0.50 |
| 79 | SKHKGGKHKGGKHKGSSGK | 0.025 |
| 80 | SHGHGGHGHGSSGK | 4.0 |

The results shown in Table 4 indicate that all of the peptides synthesized to contain two or more triplets of positively charged amino acids (a triplet containing at least one histidine residue and at least one lysine residue, but not more than 2 histidine residues or two lysine residues) demonstrated binding affinity to metal with an EC50 of less than 1 μM; whereas a peptide containing several positively charged amino acids but lacking a metal binding domain according to the present invention had comparably poorer binding affinity (SEQ ID NO:80). Several of the peptides (see, e.g., SEQ ID NOs:70-73 & 79) demonstrated high binding affinity as measured by an EC50 in a preferred range of <0.10 μM, and more preferably less than 50 nm. This high binding specificity is an improvement (in some cases, over a 10 fold improvement) over known metal binding peptides (such as those described by Sano and Shiba, J. Am. Chem. Soc., 2003, 125:14234-235) having a titanium binding EC50 of >0.10 μM. Comparing Tables 1 (illustrating the metal binding peptides isolated by phage display selections on titanium) & 4 (illustrating engineered metal binding peptides), also demonstrated is an unexpected significant increase in metal binding affinity (binding specificity for metal) which was achieved by engineering into the peptide sequence a series of two or more triplets according to the present invention.

From the amino acid sequences of the peptides illustrated in Table 4, apparent is a metal binding motif (“metal binding domain”) comprised of Z₁(Xaa)ⱼZ₂ (SEQ ID NO:2), Z₁(Xaa)ⱼZ₂(Xaa)ⱼZ (SEQ ID NO:3), and a combination thereof; wherein Z is a triplet of amino acids consisting of at least one histidine residue and at least one lysine residue, no other amino acids other than histidine residues and lysine residues, but no more than two histidine residues or no more than two lysine residues (e.g., KHK, HKH, KKH, HKK, KHH); and wherein more preferably, Z is one of HKH, KKH, or KHK, and most preferably, at least one of Z (e.g., either Z₁ or Z₂, or both of Z₁ and Z₂, in the amino acid sequence Z₁XaaXaaZ₂) is KHK, and j is preferably from 2 to 4. As illustrated in Table 4, examples of the metal binding domain include amino acid sequences

| | |
|---|---|
| KHKXaaXaaKHK, | (SEQ ID NO: 4) |
| HKHXaaXaaHKH, | (SEQ ID NO: 5) |
| KKHXaaXaaKKH, | (SEQ ID NO: 6) |
| KHKXaaXaaHKH, | (SEQ ID NO: 7) |
| HKHXaaXaaKHK, | (SEQ ID NO: 8) |
| KHKXaaXaaKHKXaaXaaKHK, | (SEQ ID NO: 9) |
| and | |
| HKHXaaXaaHKHXaaXaaHKH. | (SEQ ID NO: 10) |

### EXAMPLE 3

In this example, illustrated is the effect of spacing between triplets in the sequence of the metal binding domain according to the present invention. Synthesized were peptides which varied in the number of amino acids between triplets according to the present invention. The peptides were also synthesized to contain an amino acid linker (GSSGK portion of SEQ ID NOs:81-84) which was then biotinylated to facilitate detection and quantification. The binding assays were performed using the methods provided in Example 2 herein. As shown in Table 5, relative binding specificity (EC50) to titanium was determined and compared to the relative binding specificity of the metal binding motif having an amino acid sequence of SEQ ID NO:4 and the peptide having an amino acid sequence of SEQ ID NO:70 containing this metal binding motif (see Table 4).

TABLE 5

Spacing between triplets and effect on binding
specificity

| | | |
|---|---|---|
| SEQ ID NO. | Amino acid sequence | EC50 (μM) |

| | | |
|---|---|---|
| 81 | SKHKKHKGSSGK | 0.060 |
| 82 | SKHKGKHKGSSGK | 0.060 |
| 71 | SKHKGGKHKGSSGK | 0.026 |
| 83 | SKHKGGGKHKGSSGK | 0.075 |
| 84 | SKHKGGGGGKHKGSSGK | 0.070 |

As illustrated in Table 5, unexpectedly, the highest binding specificity is with the metal binding motif having an amino acid sequence of SEQ ID NO:4 (XaaXaa between each triplet) as compared to the metal binding domain having an amino acid sequences of any one of SEQ ID NO:42 (Xaa between each triplet), SEQ ID NO:43 (XaaXaaXaa between each triplet), and SEQ ID NO:45 (XaaXaaXaaXaaXaa between each triplet).

### EXAMPLE 4

In this embodiment, illustrated are additional characterizations of the binding specificities of examples of metal binding domains, and peptides containing the metal binding domains, according to the present invention to various substrates, such as metal (as illustrated by stainless steel, zirconium metal alloy and glass) versus binding to a polymer (as illustrated by polystyrene). Using the methods illustrated in Example 2, binding specificities for the various substrates were determined; with the results illustrated in Table 6 (Note: “none” means any binding detected was very low, and not above background binding (such as by a control peptide with no binding specificity for metal) in the binding assay; and thus, a binding curve for calculation of an EC50 could not be generated).

TABLE 6

Binding specificities for various substrates

| | | | | | |
|---|---|---|---|---|---|
| Pep- | | | | | |
| tide | | | EC50 (μM) | | EC50 |
| SEQ | | EC50 (μM) | zirconium | EC50 | (μM) |
| ID | Metal binding | stainless | metal | (μM) | Poly- |
| NO: | domain | steel | alloy | Glass | mer |

| | | | | | |
|---|---|---|---|---|---|
| 71 | SEQ ID NO: 4 | <0.5 | <0.1 | <0.5 | none |
| 77 | SEQ ID NO: 5 | <1.0 | <1.0 | <1.0 | none |
| 70 | SEQ ID NO: 6 | <0.1 | <0.1 | <0.1 | none |
| 73 | SEQ ID NO: 7 | <0.5 | <0.5 | <0.5 | none |
| 74 | SEQ ID NO: 8 | <0.5 | <0.1 | <0.5 | none |
| 75 | SEQ ID NO: 44 | <0.5 | <0.1 | <0.1 | none |
| 79 | SEQ ID NO: 9 | <0.05 | <0.05 | <0.05 | none |
| 72 | SEQ ID NO: 10 | <0.1 | <0.1 | <0.05 | None |

From the results in Table 6, it is clear that metal binding domains, and peptides containing the metal binding domains, according to the present invention have binding specificity for various metal substrates, and lack binding specificity for non-metal substrates such as a polymer. Further, in general, the metal binding peptides with the highest binding specificity (as represented by the lowest EC50) for titanium also had the highest binding affinity for metal substrates other than titanium.

### EXAMPLE 5

A metal binding peptide according to the present invention may further comprise a multimer (“polymer”) of metal binding domains according to the present invention. To illustrate this embodiment, a branched dimer (SEQ ID NO:85) and a branched tetramer (SEQ ID NO:86) were constructed using the metal binding domain consisting essentially of the amino acid sequence consisting of SEQ ID NO:9. The polymers may be illustrated by the following representation.

SEQ ID NO: 85

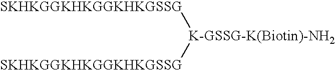

SEQ ID NO: 86

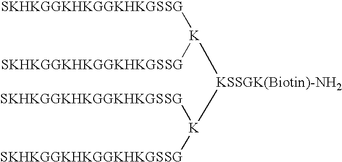

These polymers, having amino acid sequences consisting essentially of SEQ ID NOs:85 and 86, were synthesized as follows. Briefly, the polymers were built on a lysine MAP core and comprised of two and four peptide modules, respectively, of an amino acid sequence consisting essentially of SEQ ID NO:79. This core matrix was used to generate a peptide dimer and peptide tetramer using, in each branch, a monomeric peptide consisting essentially of the amino acid sequence of SEQ ID NO:79. The polymers were synthesized sequentially using solid phase chemistry on a peptide synthesizer. The synthesis was carried out at a 0.05 mmol scale which ensures maximum coupling yields during synthesis. The biotin reporter moiety was placed at the C-terminus of the molecule, and was appended by a short linker containing glycine and serine residues to the lysine core. Standard Fmoc/t-Bu chemistry was employed using AA/HBTU/HOBt/NMM (1:1:1:2) as the coupling reagents (AA is amino acid; HOBt is O-Pfp ester/1-hydroxybenzotriazole; HBTU is N-[1H-benzotriazol-1-yl)(dimethylamino)methylene]-N-methylmethanaminium hexafluorophosphate N-oxide; NMM is N-methylmorpholine). Amino acids were used in 5-10 fold excess in the synthesis cycles, and all residues were doubly, triply or even quadruply coupled depending upon the complexity of residues coupled. The coupling reactions were monitored by Kaiser ninhydrin test. The Fmoc deprotection reactions was carried out using 20% piperidine in dimethyl-formamide. Peptide cleavage from the resin was accomplished using trifluoracetic acid (TFA: H₂O:Triisopropylsilane=95:2.5:2.5) at room temperature for 4 hours. The crude product was precipitated in cold ether. The pellet obtained after centrifugation was washed thrice with cold ether and lyophilized to give a white solid as crude desired product. The crude products were analyzed by analytical high performance liquid chromatography (HPLC) on a C-18 column using mobile eluants (A=H₂O/TFA (0.1% TFA) and B=Acetonitrile/TFA (0.1% TFA). The polymers were also further analyzed by mass spectrometry for before subjecting each to final purification by HPLC. The fractions containing the desired product were pooled and lyophilized to obtain a fluffy white powder (>98% purity).

Using the methods provided in Example 2, a binding assay was performed to compare the binding specificity to titanium of the parent monomeric peptide with the polymer comprising the peptide dimer, and the polymer comprising the peptide tetramer (the structures of the dimer and tetramer are represented above). The comparison showing the binding specificities for the peptide monomer (Table 7, “SEQ ID NO:9”), the polymer comprising the peptide dimer (Table 7, “SEQ ID NO:85”), and the polymer comprising the peptide tetramer (Table 7, “SEQ ID NO:86”) are represented in Table 7.

TABLE 7
Comparison of peptide monomer to peptide polymers

| Peptide | EC50 (μM) |
|---|---|
| SEQ ID NO: 9 | 0.025 |
| SEQ ID NO: 85 | 0.020 |
| SEQ ID NO: 86 | <0.005 |

From the results in Table 7, the peptide dimer had similar high binding specificity to titanium as did the peptide monomer. However, the peptide tetramer showed at least a 5-fold increase in binding affinity for titanium as compared to the peptide monomer. Thus, binding specificities for metal may be improved by producing a polymer of a metal binding domain according to the present invention.

### EXAMPLE 6

This example illustrates peptides comprising a binding domain having a binding specificity for a pharmaceutically active agent, which can be coupled to a peptide having binding specificity for metal according to the present invention, in forming a coating composition according to the present invention.

In one embodiment, the pharmaceutically active agent is a growth factor. Thus, a coating composition according to the present invention comprises at least one peptide according to the present invention having binding specificity to metal coupled to at least one peptide having binding specificity for growth factor. Such coating composition may further comprise growth factor bound to the at least one peptide having binding specificity for the growth factor. One example of a growth factor useful with the present invention is selected from the transforming growth factor-beta family. In one embodiment, the growth factor may comprise metal morphogenetic proteins (BMP). For example, published U.S. patent application US 20060051396 (assigned to the present assignee) discloses 2 families of peptides having binding specificity for BMP. One family of BMP binders is represented by a peptide comprising the consensus sequence of GGGAWEAFSSLSGSRV (SEQ ID NO:87; which showed binding specificity for several members of the BMP family, including BMP2, BMP4, BMP5, BMP7, and BMP14); and another family of BMP binders is represented by a peptide comprising the consensus sequence of GGALGFPLKGEVVEGWA (SEQ ID NO:88). In another example, previously disclosed is a peptide which binds the growth factor transforming growth factor beta-1 (TGFβ1) and has an amino acid sequence of KRIWFIPRSSWYERA (SEQ ID NO:89).

In another embodiment, the pharmaceutically active agent is a cell (preferably, cells of a cell type). Thus, a coating composition according to the present invention comprises at least one peptide according to the present invention having binding specificity to metal coupled to at least one peptide having binding specificity for cells. Such coating composition may further comprise cells bound to the at least one peptide having binding specificity for the cells. For example, RGDX peptides (X is any amino acid; SEQ ID NO:90) have been described as binding stem cells, mesenchymal stem cells, and osteoblasts. A peptide having a sequence of ALPSTSSQMPQL (SEQ ID NO:91) has been described as binding to stem cells. In a further example, a peptide comprising the amino acid sequence of SSSCQHVSLLRPSAALGPDNCSR (SEQ ID NO:92) has binding specificity for human adipose-derived stem cells (U.S. application Ser. No. 11/649950 assigned to the present assignee), and also have bind specificity for endothelial cells.

In another embodiment, the pharmaceutically active agent is a vitamin. Thus, a coating composition according to the present invention comprises at least one peptide according to the present invention having binding specificity to metal coupled to at least one peptide having binding specificity for a vitamin. Such coating composition may further comprise the vitamin bound to the at least one peptide having binding specificity for the vitamin. For example, a peptide derived from the human Vitamin D binding protein, and having the amino acid sequence of LERGRDYEKNKVCKEFSHLGKDDFEDF (SEQ ID NO:93), has been described as binding to vitamin D sterols.

In another embodiment, the pharmaceutically active agent comprises a therapeutic drug. Thus, a coating composition according to the present invention comprises at least one peptide according to the present invention having binding specificity to metal coupled to at least one peptide having binding specificity for a therapeutic drug. Such coating composition may further comprise the therapeutic drug bound to the at least one peptide having binding specificity for the therapeutic drug. For example, as a result of using phage display to screen for peptides that bind to paclitaxel (trade name Taxol®), identified was a peptide having the amino acid sequence of HTPHPDASIQGV (SEQ ID NO:94). In another embodiment where the pharmaceutically active agent comprises a therapeutic drug, the therapeutic drug comprises an antimicrobial. Thus, a coating composition according to the present invention comprises at least one peptide according to the present invention having binding specificity to metal coupled to at least one peptide having binding specificity for a therapeutic drug comprising an antimicrobial. Such coating composition may further comprise the therapeutic drug bound to the at least one peptide having binding specificity for the therapeutic drug. For example, vancomycin and vancomycin analogs bind to bacterial cell wall peptides ending with D-Ala-D-Ala (two D-alanine residues). A peptide that mimics bacterial cell wall peptide binding to vancomycin comprises an amino acid sequence of Lys-Ala-Ala (wherein Ala is in the D form).

In another embodiment, the pharmaceutically active agent comprises a hormone. Thus, a coating composition according to the present invention comprises at least one peptide according to the present invention having binding specificity to metal coupled to at least one peptide having binding specificity for a hormone. Such coating composition may further comprise the hormone bound to the at least one peptide having binding specificity for the hormone. For example, peptides having a core amino acid sequence of VMNV (SEQ ID NO:95) have been described as binding to human growth hormone.

In another embodiment, the pharmaceutically active agent comprises a nucleic acid molecule, and more preferably, a nucleic acid molecule encoding a growth factor, therapeutic drug, hormone, or vitamin; or other nucleic acid molecule having bioactivity itself. Thus, a coating composition according to the present invention comprises at least one peptide according to the present invention having binding specificity to metal coupled to at least one peptide having binding specificity for a nucleic acid molecule. Such coating composition may further comprise the nucleic acid molecule bound to the at least one peptide having binding specificity for the nucleic acid molecule. For example, peptide having the amino acid sequence of AEDG (SEQ ID NO:96) complexes with duplex DNA comprising [poly (dA-dT): poly(dA-dT)].

Using these methods described herein, for example, a binding domain comprising a peptide according to the present invention and having binding specificity for metal may be linked to a binding domain comprising a peptide having binding specificity for a selected pharmaceutically active agent, in forming a coating composition according to the present invention. As apparent to one skilled in the art, a method of preference for linking a linker molecule to a binding domain will vary according to the reactive groups present on each molecule. Protocols for covalently linking two molecules using reactive groups are well known to one of skill in the art. As previously described herein, using methods well known to those skilled in the art, two binding domains may be coupled by a linker to form a coating composition according to the present invention by synthesizing a single contiguous peptide comprising a first binding domain, a linker comprising 3 or more amino acids (e.g., comprised of one or more of glycine and serine), and a second binding domain. The terms “first” and “second” are only used for purposes of ease of description, and is not intended to be construed as to limiting the order of the synthesis. In other words, the first binding domain may comprise a peptide having binding specificity for a selected pharmaceutically active agent, and the second binding domain may comprise a peptide having binding specificity for metal; or a first binding domain may comprise a peptide having binding specificity for metal, and a second binding domain may comprise a peptide having binding specificity for a selected pharmaceutically active agent.

### EXAMPLE 7

In this example, illustrated are methods according to the present invention: (a) a method for manufacturing a coated metal implant; (b) a method of coating a surface of metal with a peptide according to the present invention; (c) a method of coating a surface of metal with a peptide according to the present invention in providing a process selected from the group consisting of delivery of a metal binding peptide to the coated metal surface, delivery of a pharmaceutically active agent to the coated metal surface, localizing a pharmaceutically active agent to the coated metal surface, recruiting a pharmaceutically active agent to the coated metal surface, and a combination thereof; and (d) a delivery system for metal that comprises a coating composition which, when applied to metal, provides a benefit selected from the group consisting of delivery of a metal binding peptide to the coated metal surface, pharmaceutically active agent to the coated metal surface, localizing a pharmaceutically active agent to the coated metal surface, recruiting a pharmaceutically active agent to the coated metal surface, and a combination thereof.

The methods and delivery system comprise contacting at least one surface of metal with an effective amount of a peptide according to the present invention, by itself or as a component in a coating composition according to the present invention, under conditions suitable for the peptide to bind to the metal surface in producing a coating on the surface, wherein the coating composition comprises a coating composition selected from the group consisting of at least one binding domain comprising a peptide having binding specificity for metal according to the present invention; at least one binding domain comprising a peptide having binding specificity for metal according to the present invention and at least one binding domain comprising a peptide having binding specificity for a pharmaceutically active agent (wherein the at least one binding domain comprising a peptide having binding specificity for metal according to the present invention and at least one binding domain comprising a peptide having binding specificity for a pharmaceutically active agent are coupled together; preferably, via a linker); and a combination thereof. The at least one binding domain comprising a peptide having binding specificity for metal according to the present invention may be comprised of two or more peptides of the present invention linked together (e.g., linked by a multi-branched linker) and comprising of the same amino acid sequence, or may comprised of two or more peptides linked together, each comprising a different amino acid sequence.

The at least one binding domain comprising a peptide having binding specificity for a pharmaceutically active agent can comprise a single type (i.e., two or more peptides, each having binding specificity for a single type of pharmaceutically active agent, such as, for example, cells), or may comprise a plurality of types (i.e., two or more peptides, each type comprising a peptide having binding specificity for a different pharmaceutically active agent than another type; e.g., a first peptide having binding specificity for a pharmaceutically active agent comprising cells, a second peptide having binding specificity for a growth factor, etc., or a first peptide having binding specificity for a first growth factor and a second peptide having binding specificity for a second growth factor, etc.).

In these methods according to the present invention, when coating composition is contacted with the at least one surface of metal to be coated, either (a) the at least one peptide having binding specificity for a pharmaceutically active agent is bound to the pharmaceutically active agent for which it has binding specificity (for example, capture of pharmaceutically active agent of exogenous origin by peptide); or (b) the at least one peptide having binding specificity for a pharmaceutically active agent is not yet bound to the pharmaceutically active agent for which it has binding specificity such as, for example, when a metal coated with the coating composition is implanted. With respect to the latter, in a further step of coating, the coated surface metal is then contacted with a sufficient amount of pharmaceutically active agent (in vitro or in vivo), for which the at least one peptide has binding specificity, under conditions suitable so that the pharmaceutically active agent binds to the at least one peptide. In one example, coated metal may be contacted in vitro with a pharmaceutically active agent (e.g., cells and/or growth factor) which is autologous or from a donor (e.g., allogeneic or xenogeneic) for the pharmaceutically active agent can bind to the peptide comprising the coated surface of the metal, and subsequently the metal is implanted. In another example, coated metal may be implanted, wherein in vivo the coated metal is contacted with and binds to a pharmaceutically active agent (e.g., cells and/or growth factor) which is endogenously produced by the individual receiving the coated metal. By binding one or more pharmaceutically active agents to coated metal, promoted is the localization of the activity of the pharmaceutical agent to the coated metal.

Conventional processes known in the art may be used to apply the coating composition according to the present invention to the one or more surfaces of metal to be coated (in contacting the coating composition with the one or more surfaces). Depending on the formulation of metal to be coated, such processes are known to include, but are not limited to, mixing, dipping, brushing, spraying, and vapor deposition. For example, a solution or suspension comprising the coating composition may be applied through the spray nozzle of a spraying device, creating droplets that coat the surface of metal to be coated. The coated metal is allowed to dry, and may then be further processed prior to use (e.g., washed in a solution (e.g., water or isotonic buffer) to remove excess coating composition; if for in vivo use, by sterilization using any one or methods known in the art for sterilizing metal; etc.). Alternatively, where the metal comprises an implant, the coating composition and the implant may each be sterilized prior to the process of coating, and the process performed under sterile conditions.

In another process for applying the coating composition to one or more surfaces of metal to be coated, the surface of metal to be coated is dipped into a liquid (e.g., solution or suspension, aqueous or solvent) containing coating composition in an amount effective to coat metal. For example, the surface is dipped or immersed into a bath containing the coating composition. Suitable conditions for applying the coating composition include allowing the surface to be coated to remain in contact with the liquid containing the coating composition for a suitable period of time (e.g., ranging from about 5 minutes to about 12 hours; more preferably, ranging from 15 minutes to 60 at a suitable temperature (e.g., ranging from 10° C. to about 50° C.; more preferably, ranging from room temperature to 37° C.). The coated metal may then be further processed, as necessary for use (e.g., washing, sterilization, and the like). These illustrative processes for applying a coating composition to metal are not exclusive, as other coating and stabilization methods may be employed (as one of skill in the art will be able to select the compositions and methods used to fit the needs of the particular device and purpose).

Additionally, in a method according to the present invention, a coat on a metal surface comprising the coating composition may be stabilized, for example, by air drying. However, these treatments are not exclusive, and other coating and stabilization methods may be employed. Suitable coating and stabilization methods are known in the art. For example, the at least one surface of metal to be coated with the coating composition of the present invention may be pre-treated prior to the coating step so as to enhance one or more of: the binding of peptide having binding specificity for metal to be coated; and the consistency and uniformity of the coating. For example, such pretreatment may comprise etching or acid-treating the metal surface to be coated in enhancing the binding of a peptide having binding specificity for metal (e.g., by enhancing hydrophilic interactions, or the molecular adhesiveness, between the metal surface and amino acids of the peptide of the coating composition).

### EXAMPLE 8

In this example, illustrated is an example of a coating composition according to the present invention comprising at least one peptide having binding specificity for metal, coupled to at least one peptide having binding specificity for a pharmaceutically active agent; and may further comprise pharmaceutically active agent bound thereto. A metal binding peptide according to the present invention comprising an amino acid sequence consisting of SEQ ID NO:79 was biotinylated. A coating composition according to the present invention was produced by linking the metal binding peptide according to the present invention to a biotinylated peptide having binding specificity for cells (see, e.g., Example 6 herein) through a streptavidin linkage (the two different peptides added at a 1:1 ratio to streptavidin). Thus, a coating composition was formed using a linker comprising biotin and streptavidin to link at least one peptide comprising a metal binding peptide according to the present invention to at least one peptide having binding specificity for a pharmaceutically active agent.

The coating composition according to the present invention was then tested for its ability to selectively adhere cells to a metal surface. In this example, titanium disks were contacted with a buffered solution containing the coating composition at a concentration of 1 μM for 20 minutes at room temperature. As controls for non-specific binding, some disks were uncoated in the assay. 1,000,000 cells of cell line 300.19 were incubated with a green fluorescence-cell permeating dye as per the manufacturer's directions for fluorescently labeling cells. The disks were washed and 250,000 cells were added in PBS, and incubated at room temperature for 25 minutes. The disks were washed in PBS, and the cells retained on the metal substrate were visualized using epifluorescence microscopy and digital images using a digital camera. The relative fluorescence was quantitated using commercial imaging software measuring mean fluorescence intensity of each sample. The fluorescence intensity was compared between the uncoated (control) disks and the disks coated with the coating composition according to the present invention. The coating composition according to the present invention showed the ability to bind cells to the metal surface by demonstrating about a 10 fold increase in the number of cells bound to the metal disks, as compared to any of the controls.

### EXAMPLE 9

It is apparent to one skilled in the art, that based on the amino acid sequence of the peptide comprising a binding domain with binding specificity for metal in accordance with the present invention, polynucleotides (nucleic acid molecules) encoding such a peptide (or variants thereof as described herein) may be synthesized or constructed, and that such a peptide may be produced by recombinant DNA technology as a means of manufacture (e.g., in culture) and/or in vivo production by introducing such polynucleotides in vivo. For example, it is apparent to one skilled in the art that more than one polynucleotide sequence can encode a peptide according to the present invention, and that such polynucleotides may be synthesized on the bases of triplet codons known to encode the amino acids of the peptide, third base degeneracy, and selection of triplet codon usage preferred by cell-free expression system or the host cell (typically a prokaryotic cell or eukaryotic cell (e.g., bacterial cells such as E. coli; yeast cells; mammalian cells; avian cells; amphibian cells; plant cells; fish cells; and insect cells; whether located in vitro or in vivo) in which expression is desired. It would be routine for one skilled in the art to generate the degenerate variants described above, for instance, to optimize codon expression for a particular host (e.g., change codons in the bacteria mRNA to those preferred by a mammalian, plant or other bacterial host such as E. coli).

For purposes of illustration only, and not limitation, provided are SEQ ID NO:97-101 which are polynucleotides encoding amino acid sequences of SEQ ID NO:70, 72, 73, 74, and 79, respectively from which, as apparent to one skilled in the art, codon usage will generally apply to polynucleotides encoding a peptide according to the present invention which has binding specificity for metal. Thus, for example, using SEQ ID NO:97 in relation to SEQ ID NO:70, one skilled in the art could readily construct a polynucleotide encoding variants of the amino acid sequence illustrated in SEQ ID NO:70, or deduce the polynucleotide sequence encoding an amino acid sequence illustrated as SEQ ID NO:71. In a preferred embodiment of the present invention, a polynucleotide encoding an amino acid sequence of a peptide having binding specificity for metal (e.g., SEQ ID NO:79) comprises a nucleic acid molecule encoding a peptide consisting essentially of the amino acid sequence (e.g., SEQ ID NO:79) or an amino acid sequence having at least 95% identity (and more preferably, at least 90% identity) with the amino acid sequence (e.g., with SEQ ID NO:79), provided the encoded peptide contains a metal binding domain of the present invention for binding specificity for metal.

In one illustrative embodiment, provided is a recombinant vector containing a polynucelotide encoding a binding domain comprising a peptide having binding specificity for metal for use in accordance with the present invention; and its use for the recombinant production of a peptide having binding specificity for metal. In one example, the polynucleotide may be added to a cell-free expression system known in the art for producing peptides or polypeptides. In another example, the polynucleotide may be positioned in a prokaryotic expression vector so that when the peptide is produced in bacterial host cells, it is produced as a fusion protein with other amino acid sequence (e.g., which assist in purification of the peptide; or as recombinantly coupled to a surface-binding domain). For example, there are sequences known to those skilled in the art which, as part of a fusion protein with a peptide desired to be expressed, facilitates production in inclusion bodies found in the cytoplasm of the prokaryotic cell used for expression and/or assists in purification of fusion proteins containing such sequence. Inclusion bodies may be separated from other prokaryotic cellular components by methods known in the art to include denaturing agents, and fractionation (e.g., centrifugation, column chromatography, and the like). In another example, there are commercially available vectors into which is inserted a desired nucleic acid sequence of interest to be expressed as a protein or peptide such that upon expression, purification of the gene product may be accomplished using methods standard in the art.

It is apparent to one skilled in the art that a nucleic acid sequence encoding a binding domain comprising a peptide having binding specificity for metal according to the present invention can be inserted into, and become part of a, nucleic acid molecule comprising a plasmid, or vectors other than plasmids; and other expression systems can be used including, but not limited to, bacteria transformed with a bacteriophage vector, or cosmid DNA; yeast containing yeast vectors; fungi containing fungal vectors; insect cell lines infected with virus (e. g. baculovirus); and mammalian cell lines having introduced therein (e.g., transfected or electroporated with) plasmid or viral expression vectors, or infected with recombinant virus (e.g. vaccinia virus, adenovirus, adeno-associated virus, retrovirus, etc.). Successful expression of the peptide requires that either the recombinant nucleic acid molecule comprising the encoding sequence of the peptide, or the vector itself, contain the necessary control elements for transcription and translation which is compatible with, and recognized by the particular host system used for expression.

Using methods known in the art of molecular biology, including methods described above, various promoters and enhancers can be incorporated into the vector or the recombinant nucleic acid molecule comprising the encoding sequence to increase the expression of the peptide, provided that the increased expression of the peptide is compatible with (for example, non-toxic to) the particular host cell system used. As apparent to one skilled in the art, the selection of the promoter will depend on the expression system used. Promoters vary in strength, i.e., ability to facilitate transcription. Generally, for the purpose of expressing a cloned gene, it is desirable to use a strong promoter in order to obtain a high level of transcription of the gene and expression into gene product. For example, bacterial, phage, or plasmid promoters known in the art from which a high level of transcription has been observed in a host cell system comprising E. coli include the lac promoter, trp promoter, T7 promoter, recA promoter, ribosomal RNA promoter, the P.sub.R and P.sub.L promoters, lacUV5, ompF, bla, Ipp, and the like, may be used to provide transcription of the inserted nucleotide sequence encoding the synthetic peptide. Commonly used mammalian promoters in expression vectors for mammalian expression systems are the promoters from mammalian viral genes. Examples include the SV40 early promoter, mouse mammary tumor virus LTR promoter, adenovirus major late promoter, herpes simplex virus promoter, and the CMV promoter.

In the case where expression of the peptide may be lethal or detrimental to the host cells, the host cell strain/line and expression vectors may be chosen such that the action of the promoter is inhibited until specifically induced. For example, in certain operons the addition of specific inducers is necessary for efficient transcription of the inserted DNA (e.g., the lac operon is induced by the addition of lactose or isopropylthio-beta-D-galactoside (“IPTG”); trp operon is induced when tryptophan is absent in the growth media; and tetracycline can be use in mammalian expression vectors having a tet sensitive promoter). Thus, expression of the peptide may be controlled by culturing transformed or transfected cells under conditions such that the promoter controlling the expression from the encoding sequence is not induced, and when the cells reach a suitable density in the growth medium, the promoter can be induced for expression from the encoding sequence. Other control elements for efficient gene transcription or message translation are well known in the art to include enhancers, transcription or translation initiation signals, transcription termination and polyadenylation sequences, and the like.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept of the present invention; and thus, such modifications and/or adaptations are intended to be within the meaning and scope of the appended claims.

What is claimed is:

1. A peptide having formula:
(Xaa)ₘZ₁(Xaa)ⱼZ₂(Xaa)ₙ (SEQ ID NO:1), wherein:
Xaa of (Xaa)ₘ and (Xaa)ₙ is any amino acid;
Xaa of (Xaa)ⱼ is any amino acid other than lysine or histidine;
Z consists of three amino acids, with at least one histidine residue and at least one lysine residue, no other amino acids other than histidine and lysine residues, but no more than two histidine residues or no more than two lysine residues;
m is from 0 to 50; n is from 0 to 50; j is from 0 to 5; and
wherein the peptide has binding specificity for metal.

2. The peptide according to claim 1, wherein j is 2.

3. The peptide according to claim 1, wherein one or more of Z₁ and Z₂ consists of an amino acid sequence KHK.

4. The peptide according to claim 1, wherein one or more of (Xaa)ₘ and (Xaa)ₙ comprises from 0 to no more than 10 Z.

5. The peptide according to claim 1, wherein the peptide is a polymer comprising a plurality of metal binding domains consisting of Z₁(Xaa)ⱼZ₂(SEQ ID NO:2).

6. The peptide according to claim 5, wherein the polymer comprises a metal binding domain consisting of Z₁(Xaa)ⱼZ₂(Xaa)ⱼZ (SEQ ID NO:3).

7. The peptide according to claim 1, wherein the metal is selected from the group consisting of a metal represented in the Periodic Table, a metal alloy, a metal oxide, a silicon oxide, and bioactive glass, titanium, titanium alloy, stainless steel, aluminum, zirconium alloy metal substrate, and cobalt chromium alloy.

8. A composition comprising a peptide according to claim 1, and a component selected from the group consisting of pharmaceutically active agent linked to the peptide, a pharmaceutically acceptable carrier, and a combination thereof.

9. An isolated peptide consisting essentially of an amino acid sequence selected from the group consisting of KHKXaaXaaKHK (SEQ ID NO:4), HKHXaaXaaHKH (SEQ ID NO:5), KKHXaaXaaKKH (SEQ ID NO:6), KHKXaaXaaHKH (SEQ ID NO:7), HKHXaaXaaKHK (SEQ ID NO:8), KHKXaaXaaKHKXaaXaaKHK (SEQ ID NO:9), HKHXaaXaaHKHXaaXaaHKH (SEQ ID NO:10), HKHXaaXaaKKH (SEQ ID NO:11), KKHXaaXaaKHK (SEQ ID NO:12), KKHXaaXaaHKH (SEQ ID NO:13), KHKXaaXaaKKH (SEQ ID NO:14), KHKXaaXaaHKHXaaXaaKKH (SEQ ID NO:15), KHKXaaXaaKKHXaaXaaHKH (SEQ ID NO:16), KHKXaaXaaHKHXaaXaaKHK (SEQ ID NO:17), KHKXaaXaaKHKXaaXaaHKH (SEQ ID NO:18), KHKXaaXaaKKHXaaXaaKHK (SEQ ID NO:19), KHKXaaXaaKHKXaaXaaKKH (SEQ ID NO:20), KHKXaaXaaKKHXaaXaaKKH (SEQ ID NO:21), KHKXaaXaaHKHXaaXaaHKH (SEQ ID NO:22), HKHXaaXaaHKHXaaXaaKKH (SEQ ID NO:23), HKHXaaXaaKKHXaaXaaHKH (SEQ ID NO:24), HKHXaaXaaHKHXaaXaaKHK (SEQ ID NO:25), HKHXaaXaaKHKXaaXaaHKH (SEQ ID NO:26), HKHXaaXaaKHKXaaXaaKHK (SEQ ID NO:27), HKHXaaXaaKHKXaaXaaHKH (SEQ ID NO:28), HKHXaaXaaKHKXaaXaaKKH (SEQ ID NO:29), HKHXaaXaaKKHXaaXaaKKH (SEQ ID NO:30), HKHXaaXaaKKHXaaXaaKHK (SEQ ID NO:31), KKHXaaXaaHKHXaaXaaKKH (SEQ ID NO:32), KKHXaaXaaKKHXaaXaaHKH (SEQ ID NO:33), KKHXaaXaaHKHXaaXaaKHK (SEQ ID NO:34), KKHXaaXaaKHKXaaXaaHKH (SEQ ID NO:35), KKHXaaXaaKHKXaaXaaKHK (SEQ ID NO:36), KKHXaaXaaKHKXaaXaaHKH (SEQ ID NO:37), KKHXaaXaaKHKXaaXaaKKH (SEQ ID NO:38), KKHXaaXaaKKHXaaXaaKKH (SEQ ID NO:39), KKHXaaXaaHKHXaaXaaHKH (SEQ ID NO:40), KKHXaaXaaKKHXaaXaaKHK (SEQ ID NO:41), KHKXaaKHK (SEQ ID NO:42), KHKXaaXaaXaaKHK (SEQ ID NO:43), KHKXaaXaaXaaXaaKHK (SEQ ID NO:44), KHKXaaXaaXaaXaaXaaKHK (SEQ ID NO:45), SKKHGGKKHGSSGK (SEQ ID NO:70), SKHKGGKHKGSSGK (SEQ ID NO:71), SHKHGGHKHGGHKHGSSGK (SEQ ID NO:72), SKHKGGHKHGSSGK (SEQ ID NO:73), SHKHGGKHKGSSGK (SEQ ID NO:74), SKHKGGGGKHKGSSGK (SEQ ID NO:75), SHKHGGGGHKHGSSGK (SEQ ID NO:76), SHKHGGHKHGSSGK (SEQ ID NO:77), SHHKGGHHKGSSGK (SEQ ID NO:78), SKHKGGKHKGGKHKGSSGK (SEQ ID NO:79), SKHKKHKGSSGK (SEQ ID NO:81), SKHKGKHKGSSGK (SEQ ID NO:82), SKHKGGGKHKGSSGK (SEQ ID NO:83), SKHKGGGGGKHKGSSGK (SEQ ID NO:84), a dimer consisting of SEQ ID NO:85, a tetramer consisting of SEQ ID NO:86, or a combination thereof; with the proviso that Xaa is an amino acid other than lysine or histidine; a conservatively substituted variant of the peptide consisting of one or more conservative substitutions in the peptide other than for a lysine residue or histidine residue, and wherein the peptide may further be modified to comprise one or more of a terminal modification, and a modification to facilitate linking of the peptide.

10. A coating composition comprising:
(a) at least one peptide comprising of at least 8 amino acids to about 100 amino acids, wherein the peptide has the formula
(Xaa)ₘZ₁(Xaa)ⱼZ₂(Xaa)ₙ (SEQ ID NO:1), wherein
Xaa of (Xaa)ₘ and (Xaa)ₙ is any amino acid,
Xaa of (Xaa)ⱼ is any amino acid other than lysine or histidine,
Z consists of three amino acids, with at least one histidine residue and at least one lysine residue, no other amino acids other than histidine and lysine residues, but no more than two histidine residues or no more than two lysine residues,
m is from 0 to 50, n is from 0 to 50, j is from 0 to 5, and
wherein the peptide has binding specificity for metal; and
(b) at least one peptide comprising an amino acid sequence consisting of from about 3 amino acids to about 100 amino acids, which peptide binds specifically to a pharmaceutically active agent; and
wherein linked are the at least one peptide which binds specifically to metal and the at least one peptide which binds specifically to a pharmaceutically active agent.

11. The coating composition according to claim 10, wherein the at least one peptide, which binds specifically to a pharmaceutically active agent, has pharmaceutically active agent bound noncovalently thereto.

12. The coating composition according to claim 10, wherein linked covalently using a linker are the at least one peptide which binds specifically to a metal and the at least one peptide which binds specifically to a pharmaceutically active agent, and wherein the linker is selected from the group consisting of bonds of the peptides to be linked, an amino acid linker, a polymer linker, and a chemical linker.

13. The coating composition according to claim 10, wherein the metal is selected from the group consisting of a metal represented in the Periodic Table, a metal alloy, a metal oxide, a silicon oxide, and bioactive glass, titanium, titanium alloy, stainless steel, aluminum, zirconium alloy metal substrate, and cobalt chromium alloy.

14. The coating composition according to claim 10, wherein the at least one peptide having binding specificity for metal consists essentially of an amino acid sequence selected from the group consisting of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:81, SEQ ID NO:82, SEQ ID NO:83, SEQ ID NO:84, SEQ ID NO:85, SEQ ID NO:86, a conservatively substituted variant of the peptide consisting of one or more conservative substitutions in the peptide other than for a lysine residue or histidine residue, and wherein the peptide may further be modified to comprise one or more of a terminal modification, and a modification to facilitate linking of the peptide.

15. A method for coating a metal, the method comprising applying a composition according to claim 8 to the metal to be coated, in forming a coating on the metal.

16. The method according to claim 15, wherein the metal is a surface of an implant.

17. A method for coating a metal, the method comprising applying a coating composition according to claim 10 to the metal to be coated, in forming a coating on the metal.

18. The method according to claim 17, wherein the metal is a surface of an implant.

19. A method for coating a metal, the method comprising applying a coating composition according to claim 11 to the metal to be coated, in forming a coating on the metal.

20. The method according to claim 19, wherein the metal is a surface of an implant.

21. The method according to claim 20, wherein the pharmaceutically active agent is bound to the peptide which binds specifically to a pharmaceutically active agent prior to placing the implant into a subject in need of the implant.

22. The method according to claim 20, wherein the pharmaceutically active agent is bound to the peptide which binds specifically to a pharmaceutically active agent after placing the implant into a subject in need of the implant.

23. A nucleic acid molecule encoding the peptide according to claim 9.


SEQUENCE LISTING (102 sequences)

SEQ ID NO: 1 - protein, 111 aa, Artificial
  synthesized
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 2 - protein, 11 aa, Artificial
  synthesized
XXXXXXXXXXX

SEQ ID NO: 3 - protein, 19 aa, Artificial
  synthesized
XXXXXXXXXXXXXXXXXXX

SEQ ID NO: 4 - protein, 8 aa, Artificial
  synthesized
KHKXXKHK

SEQ ID NO: 5 - protein, 8 aa, Artificial
  synthesized
HKHXXHKH

SEQ ID NO: 6 - protein, 8 aa, Artificial
  synthesized
KKHXXKKH

SEQ ID NO: 7 - protein, 8 aa, Artificial
  synthesized
KHKXXHKH

SEQ ID NO: 8 - protein, 8 aa, Artificial
  synthesized
HKHXXKHK

SEQ ID NO: 9 - protein, 13 aa, Artificial
  synthesized
KHKXXKHKXXKHK

SEQ ID NO: 10 - protein, 13 aa, Artificial
  synthesized
HKHXXHKHXXHKH

SEQ ID NO: 11 - protein, 8 aa, Artificial
  synthesized
HKHXXKKH

SEQ ID NO: 12 - protein, 8 aa, Artificial
  synthesized
KKHXXKHK

SEQ ID NO: 13 - protein, 8 aa, Artificial
  synthesized
KKHXXHKH

SEQ ID NO: 14 - protein, 8 aa, Artificial
  synthesized
KHKXXKKH

SEQ ID NO: 15 - protein, 13 aa, Artificial
  synthesized
KHKXXHKHXXKKH

SEQ ID NO: 16 - protein, 13 aa, Artificial
  synthesized
KHKXXKKHXXHKH

SEQ ID NO: 17 - protein, 13 aa, Artificial
  synthesized
KHKXXHKHXXKHK

SEQ ID NO: 18 - protein, 13 aa, Artificial
  synthesized
KHKXXKHKXXHKH

SEQ ID NO: 19 - protein, 13 aa, Artificial
  synthesized
KHKXXKKHXXKHK

SEQ ID NO: 20 - protein, 13 aa, Artificial
  synthesized
KHKXXKHKXXKKH

SEQ ID NO: 21 - protein, 13 aa, Artificial
  synthesized
KHKXXKKHXXKKH

SEQ ID NO: 22 - protein, 13 aa, Artificial
  synthesized
KHKXXHKHXXHKH

SEQ ID NO: 23 - protein, 13 aa, Artificial
  synthesized
HKHXXHKHXXKKH

SEQ ID NO: 24 - protein, 13 aa, Artificial
  synthesized
HKHXXKKHXXHKH

SEQ ID NO: 25 - protein, 13 aa, Artificial
  synthesized
HKHXXHKHXXKHK

SEQ ID NO: 26 - protein, 13 aa, Artificial
  synthesized
HKHXXKHKXXHKH

SEQ ID NO: 27 - protein, 13 aa, Artificial
  synthesized
HKHXXKHKXXKHK

SEQ ID NO: 28 - protein, 13 aa, Artificial
  synthesized
HKHXXKHKXXHKH

SEQ ID NO: 29 - protein, 13 aa, Artificial
  synthesized
HKHXXKHKXXKKH

SEQ ID NO: 30 - protein, 13 aa, Artificial
  synthesized
HKHXXKKHXXKKH

SEQ ID NO: 31 - protein, 13 aa, Artificial
  synthesized
HKHXXKKHXXKHK

SEQ ID NO: 32 - protein, 13 aa, Artificial
  synthesized
KKHXXHKHXXKKH

SEQ ID NO: 33 - protein, 13 aa, Artificial
  synthesized
KKHXXKKHXXHKH

SEQ ID NO: 34 - protein, 13 aa, Artificial
  synthesized
KKHXXHKHXXKHK

SEQ ID NO: 35 - protein, 13 aa, Artificial
  synthesized
KKHXXKHKXXHKH

SEQ ID NO: 36 - protein, 13 aa, Artificial
  synthesized
KKHXXKHKXXKHK

SEQ ID NO: 37 - protein, 13 aa, Artificial
  synthesized
KKHXXKHKXXHKH

SEQ ID NO: 38 - protein, 13 aa, Artificial
  synthesized
KKHXXKHKXXKKH

SEQ ID NO: 39 - protein, 13 aa, Artificial
  synthesized
KKHXXKKHXXKKH

SEQ ID NO: 40 - protein, 13 aa, Artificial
  synthesized
KKHXXHKHXXHKH

SEQ ID NO: 41 - protein, 13 aa, Artificial
  synthesized
KKHXXKKHXXKHK

SEQ ID NO: 42 - protein, 7 aa, Artificial
  synthesized
KHKXKHK

SEQ ID NO: 43 - protein, 9 aa, Artificial
  synthesized
KHKXXXKHK

SEQ ID NO: 44 - protein, 10 aa, Artificial
  synthesized
KHKXXXXKHK

SEQ ID NO: 45 - protein, 11 aa, Artificial
  synthesized
KHKXXXXXKHK

SEQ ID NO: 46 - protein, 17 aa, Artificial
  synthesized
SSHKHPVTPRFFVVESR

SEQ ID NO: 47 - protein, 22 aa, Artificial
  synthesized
SSCNCYVTPNLLKHKCYKICSR

SEQ ID NO: 48 - protein, 22 aa, Artificial
  synthesized
SSCSHNHHKLTAKHQVAHKCSR

SEQ ID NO: 49 - protein, 22 aa, Artificial
  synthesized
SSCDQNDIFYTSKKSHKSHCSR

SEQ ID NO: 50 - protein, 22 aa, Artificial
  synthesized
SSSSDVYLVSHKHHLTRHNSSR

SEQ ID NO: 51 - protein, 22 aa, Artificial
  synthesized
SSSDKCHKHWYCYESKYGGSSR

SEQ ID NO: 52 - protein, 14 aa, Artificial
  synthesized
HHKLKHQMLHLNGG

SEQ ID NO: 53 - protein, 14 aa, Artificial
  synthesized
GHHHKKDQLPQLGG

SEQ ID NO: 54 - protein, 22 aa, Artificial
  synthesized
SSCKHDSEFIKKHVHAVKKCSR

SEQ ID NO: 55 - protein, 22 aa, Artificial
  synthesized
SSCHDHSNKYLKSWKHQQNCSR

SEQ ID NO: 56 - protein, 22 aa, Artificial
  synthesized
SSSYFNLGLVKHNHVRHHDSSR

SEQ ID NO: 57 - protein, 22 aa, Artificial
  synthesized
SSCHHLKHNTHKESKMHHECSR

SEQ ID NO: 58 - protein, 15 aa, Artificial
  synthesized
SSVNKMNRLWEPLSR

SEQ ID NO: 59 - protein, 16 aa, Artificial
  synthesized
SHKHPVTPRFFVVESK

SEQ ID NO: 60 - protein, 16 aa, Artificial
  synthesized
SHKHPVTPRGGVVESK

SEQ ID NO: 61 - protein, 16 aa, Artificial
  synthesized
SHKHGGGGRFFVVESK

SEQ ID NO: 62 - protein, 16 aa, Artificial
  synthesized
SHKHPVTPRGGGGESK

SEQ ID NO: 63 - protein, 16 aa, Artificial
  synthesized
SHKHPVTPGFFVVESK

SEQ ID NO: 64 - protein, 16 aa, Artificial
  synthesized
SGGGPVTPRFFVVESK

SEQ ID NO: 65 - protein, 16 aa, Artificial
  synthesized
SHKHPVTPRFFVVYSK

SEQ ID NO: 66 - protein, 16 aa, Artificial
  synthesized
SHKHPVTPRFFVVKSK

SEQ ID NO: 67 - protein, 16 aa, Artificial
  synthesized
SHKHPVTPRFFVVVSK

SEQ ID NO: 68 - protein, 16 aa, Artificial
  synthesized
SHKHPVTPRFFVVGSK

SEQ ID NO: 69 - protein, 16 aa, Artificial
  synthesized
SHKHPVTPRFFVVNSK

SEQ ID NO: 70 - protein, 14 aa, Artificial
  synthesized
SKKHGGKKHGSSGK

SEQ ID NO: 71 - protein, 14 aa, Artificial
  synthesized
SKHKGGKHKGSSGK

SEQ ID NO: 72 - protein, 19 aa, Artificial
  synthesized
SHKHGGHKHGGHKHGSSGK

SEQ ID NO: 73 - protein, 14 aa, Artificial
  synthesized
SKHKGGHKHGSSGK

SEQ ID NO: 74 - protein, 14 aa, Artificial
  synthesized
SHKHGGKHKGSSGK

SEQ ID NO: 75 - protein, 16 aa, Artificial
  synthesized
SKHKGGGGKHKGSSGK

SEQ ID NO: 76 - protein, 16 aa, Artificial
  synthesized
SHKHGGGGHKHGSSGK

SEQ ID NO: 77 - protein, 14 aa, Artificial
  synthesized
SHKHGGHKHGSSGK

SEQ ID NO: 78 - protein, 14 aa, Artificial
  synthesized
SHHKGGHHKGSSGK

SEQ ID NO: 79 - protein, 19 aa, Artificial
  synthesized
SKHKGGKHKGGKHKGSSGK

SEQ ID NO: 80 - protein, 14 aa, Artificial
  synthesized
SHGHGGHGHGSSGK

SEQ ID NO: 81 - protein, 12 aa, Artificial
  synthesized
SKHKKHKGSSGK

SEQ ID NO: 82 - protein, 13 aa, Artificial
  synthesized
SKHKGKHKGSSGK

SEQ ID NO: 83 - protein, 15 aa, Artificial
  synthesized
SKHKGGGKHKGSSGK

SEQ ID NO: 84 - protein, 17 aa, Artificial
  synthesized
SKHKGGGGGKHKGSSGK

SEQ ID NO: 85 - protein, 23 aa, Artificial
  synthesized
SKHKGGKHKGGKHKGSSGKGSSG

SEQ ID NO: 86 - protein, 24 aa, Artificial
  synthesized
SKHKGGKHKGGKHKGSSGKKSSGK

SEQ ID NO: 87 - protein, 16 aa, Artificial
  synthesized
GGGAWEAFSSLSGSRV

SEQ ID NO: 88 - protein, 17 aa, Artificial
  synthesized
GGALGFPLKGEVVEGWA

SEQ ID NO: 89 - protein, 15 aa, Artificial
  synthesized
KRIWFIPRSSWYERA

SEQ ID NO: 90 - protein, 4 aa, Artificial
  synthesized
RGDX

SEQ ID NO: 91 - protein, 12 aa, Artificial
  synthesized
ALPSTSSQMPQL

SEQ ID NO: 92 - protein, 23 aa, Artificial
  synthesized
SSSCQHVSLLRPSAALGPDNCSR

SEQ ID NO: 93 - protein, 27 aa, Artificial
  synthesized
LERGRDYEKNKVCKEFSHLGKDDFEDF

SEQ ID NO: 94 - protein, 12 aa, Artificial
  synthesized
HTPHPDASIQGV

SEQ ID NO: 95 - protein, 4 aa, Artificial
  synthesized
VMNV

SEQ ID NO: 96 - protein, 4 aa, Artificial
  synthesized
AEDG

SEQ ID NO: 97 - DNA, 27 bp, Artificial
  synthesized
agcaagaagcacggcggcaagaagcac

SEQ ID NO: 98 - DNA, 42 bp, Artificial
  synthesized
tctcataaacatggtggtcataaacatggtggtcataaacat

SEQ ID NO: 99 - DNA, 27 bp, Artificial
  synthesized
tcaaaacataaaggaggacataaacat

SEQ ID NO: 100 - DNA, 28 bp, Artificial
  synthesized
tcgcataaacatggggggaaacataaaa

SEQ ID NO: 101 - DNA, 42 bp, Artificial
  synthesized
tctaaacataaaggtggtaaacataaaggtggtaaacataaa

SEQ ID NO: 102 - protein, 50 aa, Artificial
  synthesized
XXXXXXXXXXXXXXXXXXXXXXXXXXXXXXXXXXXXXXXXXXXXXXXXXX